SEQUENCE LISTING (197 sequences)

SEQ ID NO: 1 - DNA, 32 bp, Artificial Sequence
  Description of Artificial Sequence Synthetic oligonucleotide
tttttttaagcagtggtatcaacgcagagtac

SEQ ID NO: 2 - DNA, 32 bp, Artificial Sequence; identical to SEQ ID NO: 1
  Description of Artificial Sequence Synthetic oligonucleotide

SEQ ID NO: 3 - DNA, 72 bp, Artificial Sequence
  Description of Artificial Sequence Synthetic oligonucleotide
  modified_base (35)..(42): a, c, t, g, unknown or other
aagcagtggtatcaacgcagagtacttaaggcctnnnnnnnntttttttttttttttttttttttttttttt

SEQ ID NO: 4 - DNA, 72 bp, Artificial Sequence
  Description of Artificial Sequence Synthetic oligonucleotide
  modified_base (35)..(42): a, c, t, g, unknown or other
aagcagtggtatcaacgcagagtacaggacatgtnnnnnnnntttttttttttttttttttttttttttttt

SEQ ID NO: 5 - DNA, 72 bp, Artificial Sequence
  Description of Artificial Sequence Synthetic oligonucleotide
  modified_base (35)..(42): a, c, t, g, unknown or other
aagcagtggtatcaacgcagagtacaccaggcttnnnnnnnntttttttttttttttttttttttttttttt

SEQ ID NO: 6 - DNA, 72 bp, Artificial Sequence
  Description of Artificial Sequence Synthetic oligonucleotide
  modified_base (35)..(42): a, c, t, g, unknown or other
aagcagtggtatcaacgcagagtaccaggaatcgnnnnnnnntttttttttttttttttttttttttttttt

SEQ ID NO: 7 - DNA, 72 bp, Artificial Sequence
  Description of Artificial Sequence Synthetic oligonucleotide
  modified_base (35)..(42): a, c, t, g, unknown or other
aagcagtggtatcaacgcagagtacgatccttcgnnnnnnnntttttttttttttttttttttttttttttt

SEQ ID NO: 8 - DNA, 72 bp, Artificial Sequence
  Description of Artificial Sequence Synthetic oligonucleotide
  modified_base (35)..(42): a, c, t, g, unknown or other
aagcagtggtatcaacgcagagtacccgtcatatnnnnnnnntttttttttttttttttttttttttttttt

SEQ ID NO: 9 - DNA, 72 bp, Artificial Sequence
  Description of Artificial Sequence Synthetic oligonucleotide
  modified_base (35)..(42): a, c, t, g, unknown or other
aagcagtggtatcaacgcagagtacagtccacaannnnnnnntttttttttttttttttttttttttttttt

SEQ ID NO: 10 - DNA, 72 bp, Artificial Sequence
  Description of Artificial Sequence Synthetic oligonucleotide
  modified_base (35)..(42): a, c, t, g, unknown or other
aagcagtggtatcaacgcagagtaccatcagcctnnnnnnnntttttttttttttttttttttttttttttt

SEQ ID NO: 11 - DNA, 72 bp, Artificial Sequence
  Description of Artificial Sequence Synthetic oligonucleotide
  modified_base (35)..(42): a, c, t, g, unknown or other
aagcagtggtatcaacgcagagtacttctatggcnnnnnnnntttttttttttttttttttttttttttttt

SEQ ID NO: 12 - DNA, 72 bp, Artificial Sequence
  Description of Artificial Sequence Synthetic oligonucleotide
  modified_base (35)..(42): a, c, t, g, unknown or other
aagcagtggtatcaacgcagagtaccctcgtaagnnnnnnnntttttttttttttttttttttttttttttt

SEQ ID NO: 13 - DNA, 72 bp, Artificial Sequence
  Description of Artificial Sequence Synthetic oligonucleotide
  modified_base (35)..(42): a, c, t, g, unknown or other
aagcagtggtatcaacgcagagtactcgcaattcnnnnnnnntttttttttttttttttttttttttttttt

SEQ ID NO: 14 - DNA, 72 bp, Artificial Sequence
  Description of Artificial Sequence Synthetic oligonucleotide
  modified_base (35)..(42): a, c, t, g, unknown or other
aagcagtggtatcaacgcagagtaccttggtgcannnnnnnntttttttttttttttttttttttttttttt

SEQ ID NO: 15 - DNA, 72 bp, Artificial Sequence
  Description of Artificial Sequence Synthetic oligonucleotide
  modified_base (35)..(42): a, c, t, g, unknown or other
aagcagtggtatcaacgcagagtacagaagcttcnnnnnnnntttttttttttttttttttttttttttttt

SEQ ID NO: 16 - DNA, 72 bp, Artificial Sequence
  Description of Artificial Sequence Synthetic oligonucleotide
  modified_base (35)..(42): a, c, t, g, unknown or other
aagcagtggtatcaacgcagagtactgttggaggnnnnnnnntttttttttttttttttttttttttttttt

SEQ ID NO: 17 - DNA, 72 bp, Artificial Sequence
  Description of Artificial Sequence Synthetic oligonucleotide
  modified_base (35)..(42): a, c, t, g, unknown or other
aagcagtggtatcaacgcagagtacccacattcgnnnnnnnntttttttttttttttttttttttttttttt

SEQ ID NO: 18 - DNA, 72 bp, Artificial Sequence
  Description of Artificial Sequence Synthetic oligonucleotide
  modified_base (35)..(42): a, c, t, g, unknown or other
aagcagtggtatcaacgcagagtaccaatggtcgnnnnnnnntttttttttttttttttttttttttttttt

SEQ ID NO: 19 - DNA, 72 bp, Artificial Sequence
  Description of Artificial Sequence Synthetic oligonucleotide
  modified_base (35)..(42): a, c, t, g, unknown or other
aagcagtggtatcaacgcagagtacatatgcggtnnnnnnnntttttttttttttttttttttttttttttt

SEQ ID NO: 20 - DNA, 72 bp, Artificial Sequence
  Description of Artificial Sequence Synthetic oligonucleotide
  modified_base (35)..(42): a, c, t, g, unknown or other
aagcagtggtatcaacgcagagtacctgtaacagnnnnnnnntttttttttttttttttttttttttttttt

SEQ ID NO: 21 - DNA, 72 bp, Artificial Sequence
  Description of Artificial Sequence Synthetic oligonucleotide
  modified_base (35)..(42): a, c, t, g, unknown or other
aagcagtggtatcaacgcagagtacttcctcaagnnnnnnnntttttttttttttttttttttttttttttt

SEQ ID NO: 22 - DNA, 72 bp, Artificial Sequence
  Description of Artificial Sequence Synthetic oligonucleotide
  modified_base (35)..(42): a, c, t, g, unknown or other
aagcagtggtatcaacgcagagtaccaagttggtnnnnnnnntttttttttttttttttttttttttttttt

SEQ ID NO: 23 - DNA, 72 bp, Artificial Sequence
  Description of Artificial Sequence Synthetic oligonucleotide
  modified_base (35)..(42): a, c, t, g, unknown or other
aagcagtggtatcaacgcagagtactcgtgaggtnnnnnnnntttttttttttttttttttttttttttttt

SEQ ID NO: 24 - DNA, 72 bp, Artificial Sequence
  Description of Artificial Sequence Synthetic oligonucleotide
  modified_base (35)..(42): a, c, t, g, unknown or other
aagcagtggtatcaacgcagagtacacttcggaannnnnnnntttttttttttttttttttttttttttttt

SEQ ID NO: 25 - DNA, 72 bp, Artificial Sequence
  Description of Artificial Sequence Synthetic oligonucleotide
  modified_base (35)..(42): a, c, t, g, unknown or other
aagcagtggtatcaacgcagagtacactgcctgtnnnnnnnntttttttttttttttttttttttttttttt

SEQ ID NO: 26 - DNA, 72 bp, Artificial Sequence
  Description of Artificial Sequence Synthetic oligonucleotide
  modified_base (35)..(42): a, c, t, g, unknown or other
aagcagtggtatcaacgcagagtacgaagtctcgnnnnnnnntttttttttttttttttttttttttttttt

SEQ ID NO: 27 - DNA, 72 bp, Artificial Sequence
  Description of Artificial Sequence Synthetic oligonucleotide
  modified_base (35)..(42): a, c, t, g, unknown or other
aagcagtggtatcaacgcagagtactcctggaatnnnnnnnntttttttttttttttttttttttttttttt

SEQ ID NO: 28 - DNA, 72 bp, Artificial Sequence
  Description of Artificial Sequence Synthetic oligonucleotide
  modified_base (35)..(42): a, c, t, g, unknown or other
aagcagtggtatcaacgcagagtactctcttcggnnnnnnnntttttttttttttttttttttttttttttt

SEQ ID NO: 29 - DNA, 72 bp, Artificial Sequence
  Description of Artificial Sequence Synthetic oligonucleotide
  modified_base (35)..(42): a, c, t, g, unknown or other
aagcagtggtatcaacgcagagtacagccatggannnnnnnntttttttttttttttttttttttttttttt

SEQ ID NO: 30 - DNA, 72 bp, Artificial Sequence
  Description of Artificial Sequence Synthetic oligonucleotide
  modified_base (35)..(42): a, c, t, g, unknown or other
aagcagtggtatcaacgcagagtacctctgttgtnnnnnnnntttttttttttttttttttttttttttttt

SEQ ID NO: 31 - DNA, 72 bp, Artificial Sequence
  Description of Artificial Sequence Synthetic oligonucleotide
  modified_base (35)..(42): a, c, t, g, unknown or other
aagcagtggtatcaacgcagagtacgctacacatnnnnnnnntttttttttttttttttttttttttttttt

SEQ ID NO: 32 - DNA, 72 bp, Artificial Sequence
  Description of Artificial Sequence Synthetic oligonucleotide
  modified_base (35)..(42): a, c, t, g, unknown or other
aagcagtggtatcaacgcagagtaccgtatccatnnnnnnnntttttttttttttttttttttttttttttt

SEQ ID NO: 33 - DNA, 72 bp, Artificial Sequence
  Description of Artificial Sequence Synthetic oligonucleotide
  modified_base (35)..(42): a, c, t, g, unknown or other
aagcagtggtatcaacgcagagtactgtccttgtnnnnnnnntttttttttttttttttttttttttttttt

SEQ ID NO: 34 - DNA, 72 bp, Artificial Sequence
  Description of Artificial Sequence Synthetic oligonucleotide
  modified_base (35)..(42): a, c, t, g, unknown or other
aagcagtggtatcaacgcagagtacttggcacgannnnnnnntttttttttttttttttttttttttttttt

SEQ ID NO: 35 - DNA, 72 bp, Artificial Sequence
  Description of Artificial Sequence Synthetic oligonucleotide
  modified_base (35)..(42): a, c, t, g, unknown or other
aagcagtggtatcaacgcagagtacggcgaacttnnnnnnnntttttttttttttttttttttttttttttt

SEQ ID NO: 36 - DNA, 72 bp, Artificial Sequence
  Description of Artificial Sequence Synthetic oligonucleotide
  modified_base (35)..(42): a, c, t, g, unknown or other
aagcagtggtatcaacgcagagtacgtatgttccnnnnnnnntttttttttttttttttttttttttttttt

SEQ ID NO: 37 - DNA, 72 bp, Artificial Sequence
  Description of Artificial Sequence Synthetic oligonucleotide
  modified_base (35)..(42): a, c, t, g, unknown or other
aagcagtggtatcaacgcagagtacttaggacacnnnnnnnntttttttttttttttttttttttttttttt

SEQ ID NO: 38 - DNA, 72 bp, Artificial Sequence
  Description of Artificial Sequence Synthetic oligonucleotide
  modified_base (35)..(42): a, c, t, g, unknown or other
aagcagtggtatcaacgcagagtactcggaccttnnnnnnnntttttttttttttttttttttttttttttt

SEQ ID NO: 39 - DNA, 72 bp, Artificial Sequence
  Description of Artificial Sequence Synthetic oligonucleotide
  modified_base (35)..(42): a, c, t, g, unknown or other
aagcagtggtatcaacgcagagtacgcatcttgtnnnnnnnntttttttttttttttttttttttttttttt

SEQ ID NO: 40 - DNA, 72 bp, Artificial Sequence
  Description of Artificial Sequence Synthetic oligonucleotide
  modified_base (35)..(42): a, c, t, g, unknown or other
aagcagtggtatcaacgcagagtaccttaggagtnnnnnnnntttttttttttttttttttttttttttttt

SEQ ID NO: 41 - DNA, 72 bp, Artificial Sequence
  Description of Artificial Sequence Synthetic oligonucleotide
  modified_base (35)..(42): a, c, t, g, unknown or other
aagcagtggtatcaacgcagagtacctcagctacnnnnnnnntttttttttttttttttttttttttttttt

SEQ ID NO: 42 - DNA, 72 bp, Artificial Sequence
  Description of Artificial Sequence Synthetic oligonucleotide
  modified_base (35)..(42): a, c, t, g, unknown or other
aagcagtggtatcaacgcagagtacgcaggtattnnnnnnnntttttttttttttttttttttttttttttt

SEQ ID NO: 43 - DNA, 72 bp, Artificial Sequence
  Description of Artificial Sequence Synthetic oligonucleotide
  modified_base (35)..(42): a, c, t, g, unknown or other
aagcagtggtatcaacgcagagtactgcgtaagcnnnnnnnntttttttttttttttttttttttttttttt

SEQ ID NO: 44 - DNA, 72 bp, Artificial Sequence
  Description of Artificial Sequence Synthetic oligonucleotide
  modified_base (35)..(42): a, c, t, g, unknown or other
aagcagtggtatcaacgcagagtacattcctctcnnnnnnnntttttttttttttttttttttttttttttt

SEQ ID NO: 45 - DNA, 72 bp, Artificial Sequence
  Description of Artificial Sequence Synthetic oligonucleotide
  modified_base (35)..(42): a, c, t, g, unknown or other
aagcagtggtatcaacgcagagtactggaacgtcnnnnnnnntttttttttttttttttttttttttttttt

SEQ ID NO: 46 - DNA, 72 bp, Artificial Sequence
  Description of Artificial Sequence Synthetic oligonucleotide
  modified_base (35)..(42): a, c, t, g, unknown or other
aagcagtggtatcaacgcagagtacggattccttnnnnnnnntttttttttttttttttttttttttttttt

SEQ ID NO: 47 - DNA, 72 bp, Artificial Sequence
  Description of Artificial Sequence Synthetic oligonucleotide
  modified_base (35)..(42): a, c, t, g, unknown or other
aagcagtggtatcaacgcagagtacctacgccttnnnnnnnntttttttttttttttttttttttttttttt

SEQ ID NO: 48 - DNA, 72 bp, Artificial Sequence
  Description of Artificial Sequence Synthetic oligonucleotide
  modified_base (35)..(42): a, c, t, g, unknown or other
aagcagtggtatcaacgcagagtactgaggcgttnnnnnnnntttttttttttttttttttttttttttttt

SEQ ID NO: 49 - DNA, 72 bp, Artificial Sequence
  Description of Artificial Sequence Synthetic oligonucleotide
  modified_base (35)..(42): a, c, t, g, unknown or other
aagcagtggtatcaacgcagagtactagacgcacnnnnnnnntttttttttttttttttttttttttttttt

SEQ ID NO: 50 - DNA, 72 bp, Artificial Sequence
  Description of Artificial Sequence Synthetic oligonucleotide
  modified_base (35)..(42): a, c, t, g, unknown or other
aagcagtggtatcaacgcagagtactggtccgaannnnnnnntttttttttttttttttttttttttttttt

SEQ ID NO: 51 - DNA, 72 bp, Artificial Sequence
  Description of Artificial Sequence Synthetic oligonucleotide
  modified_base (35)..(42): a, c, t, g, unknown or other
aagcagtggtatcaacgcagagtacttcaccatcnnnnnnnntttttttttttttttttttttttttttttt

SEQ ID NO: 52 - DNA, 72 bp, Artificial Sequence
  Description of Artificial Sequence Synthetic oligonucleotide
  modified_base (35)..(42): a, c, t, g, unknown or other
aagcagtggtatcaacgcagagtacgtcgataacnnnnnnnntttttttttttttttttttttttttttttt

SEQ ID NO: 53 - DNA, 72 bp, Artificial Sequence
  Description of Artificial Sequence Synthetic oligonucleotide
  modified_base (35)..(42): a, c, t, g, unknown or other
aagcagtggtatcaacgcagagtacctgagatggnnnnnnnntttttttttttttttttttttttttttttt

SEQ ID NO: 54 - DNA, 72 bp, Artificial Sequence
  Description of Artificial Sequence Synthetic oligonucleotide
  modified_base (35)..(42): a, c, t, g, unknown or other
aagcagtggtatcaacgcagagtacttagcctgcnnnnnnnntttttttttttttttttttttttttttttt

SEQ ID NO: 55 - DNA, 72 bp, Artificial Sequence
  Description of Artificial Sequence Synthetic oligonucleotide
  modified_base (35)..(42): a, c, t, g, unknown or other
aagcagtggtatcaacgcagagtaccagttgtacnnnnnnnntttttttttttttttttttttttttttttt

SEQ ID NO: 56 - DNA, 72 bp, Artificial Sequence
  Description of Artificial Sequence Synthetic oligonucleotide
  modified_base (35)..(42): a, c, t, g, unknown or other
aagcagtggtatcaacgcagagtacccaactgaannnnnnnntttttttttttttttttttttttttttttt

SEQ ID NO: 57 - DNA, 72 bp, Artificial Sequence
  Description of Artificial Sequence Synthetic oligonucleotide
  modified_base (35)..(42): a, c, t, g, unknown or other
aagcagtggtatcaacgcagagtacctgaccaatnnnnnnnntttttttttttttttttttttttttttttt

SEQ ID NO: 58 - DNA, 72 bp, Artificial Sequence
  Description of Artificial Sequence Synthetic oligonucleotide
  modified_base (35)..(42): a, c, t, g, unknown or other
aagcagtggtatcaacgcagagtaccgatagctgnnnnnnnntttttttttttttttttttttttttttttt

SEQ ID NO: 59 - DNA, 72 bp, Artificial Sequence
  Description of Artificial Sequence Synthetic oligonucleotide
  modified_base (35)..(42): a, c, t, g, unknown or other
aagcagtggtatcaacgcagagtacgataaggagnnnnnnnntttttttttttttttttttttttttttttt

SEQ ID NO: 60 - DNA, 72 bp, Artificial Sequence
  Description of Artificial Sequence Synthetic oligonucleotide
  modified_base (35)..(42): a, c, t, g, unknown or other
aagcagtggtatcaacgcagagtacacagcgagannnnnnnntttttttttttttttttttttttttttttt

SEQ ID NO: 61 - DNA, 72 bp, Artificial Sequence
  Description of Artificial Sequence Synthetic oligonucleotide
  modified_base (35)..(42): a, c, t, g, unknown or other
aagcagtggtatcaacgcagagtacactcattgcnnnnnnnntttttttttttttttttttttttttttttt

SEQ ID NO: 62 - DNA, 72 bp, Artificial Sequence
  Description of Artificial Sequence Synthetic oligonucleotide
  modified_base (35)..(42): a, c, t, g, unknown or other
aagcagtggtatcaacgcagagtacaagagtgcgnnnnnnnntttttttttttttttttttttttttttttt

SEQ ID NO: 63 - DNA, 72 bp, Artificial Sequence
  Description of Artificial Sequence Synthetic oligonucleotide
  modified_base (35)..(42): a, c, t, g, unknown or other
aagcagtggtatcaacgcagagtactaaccgttcnnnnnnnntttttttttttttttttttttttttttttt

SEQ ID NO: 64 - DNA, 72 bp, Artificial Sequence
  Description of Artificial Sequence Synthetic oligonucleotide
  modified_base (35)..(42): a, c, t, g, unknown or other
aagcagtggtatcaacgcagagtacgttacaagcnnnnnnnntttttttttttttttttttttttttttttt

SEQ ID NO: 65 - DNA, 72 bp, Artificial Sequence
  Description of Artificial Sequence Synthetic oligonucleotide
  modified_base (35)..(42): a, c, t, g, unknown or other
aagcagtggtatcaacgcagagtaccaagcgattnnnnnnnntttttttttttttttttttttttttttttt

SEQ ID NO: 66 - DNA, 72 bp, Artificial Sequence
  Description of Artificial Sequence Synthetic oligonucleotide
  modified_base (35)..(42): a, c, t, g, unknown or other
aagcagtggtatcaacgcagagtacggaattgccnnnnnnnntttttttttttttttttttttttttttttt

SEQ ID NO: 67 - DNA, 72 bp, Artificial Sequence
  Description of Artificial Sequence Synthetic oligonucleotide
  modified_base (35)..(42): a, c, t, g, unknown or other
aagcagtggtatcaacgcagagtacgatcgtcttnnnnnnnntttttttttttttttttttttttttttttt

SEQ ID NO: 68 - DNA, 72 bp, Artificial Sequence
  Description of Artificial Sequence Synthetic oligonucleotide
  modified_base (35)..(42): a, c, t, g, unknown or other
aagcagtggtatcaacgcagagtactagcctatgnnnnnnnntttttttttttttttttttttttttttttt

SEQ ID NO: 69 - DNA, 72 bp, Artificial Sequence
  Description of Artificial Sequence Synthetic oligonucleotide
  modified_base (35)..(42): a, c, t, g, unknown or other
aagcagtggtatcaacgcagagtacacacggactnnnnnnnntttttttttttttttttttttttttttttt

SEQ ID NO: 70 - DNA, 72 bp, Artificial Sequence
  Description of Artificial Sequence Synthetic oligonucleotide
  modified_base (35)..(42): a, c, t, g, unknown or other
aagcagtggtatcaacgcagagtactacagagtcnnnnnnnntttttttttttttttttttttttttttttt

SEQ ID NO: 71 - DNA, 72 bp, Artificial Sequence
  Description of Artificial Sequence Synthetic oligonucleotide
  modified_base (35)..(42): a, c, t, g, unknown or other
aagcagtggtatcaacgcagagtacttcgaggttnnnnnnnntttttttttttttttttttttttttttttt

SEQ ID NO: 72 - DNA, 72 bp, Artificial Sequence
  Description of Artificial Sequence Synthetic oligonucleotide
  modified_base (35)..(42): a, c, t, g, unknown or other
aagcagtggtatcaacgcagagtacattggccatnnnnnnnntttttttttttttttttttttttttttttt

SEQ ID NO: 73 - DNA, 72 bp, Artificial Sequence
  Description of Artificial Sequence Synthetic oligonucleotide
  modified_base (35)..(42): a, c, t, g, unknown or other
aagcagtggtatcaacgcagagtaccatctcgcannnnnnnntttttttttttttttttttttttttttttt

SEQ ID NO: 74 - DNA, 72 bp, Artificial Sequence
  Description of Artificial Sequence Synthetic oligonucleotide
  modified_base (35)..(42): a, c, t, g, unknown or other
aagcagtggtatcaacgcagagtacccgaattgtnnnnnnnntttttttttttttttttttttttttttttt

SEQ ID NO: 75 - DNA, 72 bp, Artificial Sequence
  Description of Artificial Sequence Synthetic oligonucleotide
  modified_base (35)..(42): a, c, t, g, unknown or other
aagcagtggtatcaacgcagagtacccttgtgttnnnnnnnntttttttttttttttttttttttttttttt

SEQ ID NO: 76 - DNA, 72 bp, Artificial Sequence
  Description of Artificial Sequence Synthetic oligonucleotide
  modified_base (35)..(42): a, c, t, g, unknown or other
aagcagtggtatcaacgcagagtacggccattatnnnnnnnntttttttttttttttttttttttttttttt

SEQ ID NO: 77 - DNA, 72 bp, Artificial Sequence
  Description of Artificial Sequence Synthetic oligonucleotide
  modified_base (35)..(42): a, c, t, g, unknown or other
aagcagtggtatcaacgcagagtaccacactaggnnnnnnnntttttttttttttttttttttttttttttt

SEQ ID NO: 78 - DNA, 72 bp, Artificial Sequence
  Description of Artificial Sequence Synthetic oligonucleotide
  modified_base (35)..(42): a, c, t, g, unknown or other
aagcagtggtatcaacgcagagtactggttgtcgnnnnnnnntttttttttttttttttttttttttttttt

SEQ ID NO: 79 - DNA, 72 bp, Artificial Sequence
  Description of Artificial Sequence Synthetic oligonucleotide
  modified_base (35)..(42): a, c, t, g, unknown or other
aagcagtggtatcaacgcagagtaccaccatgtgnnnnnnnntttttttttttttttttttttttttttttt

SEQ ID NO: 80 - DNA, 72 bp, Artificial Sequence
  Description of Artificial Sequence Synthetic oligonucleotide
  modified_base (35)..(42): a, c, t, g, unknown or other
aagcagtggtatcaacgcagagtacggcttggatnnnnnnnntttttttttttttttttttttttttttttt

SEQ ID NO: 81 - DNA, 72 bp, Artificial Sequence
  Description of Artificial Sequence Synthetic oligonucleotide
  modified_base (35)..(42): a, c, t, g, unknown or other
aagcagtggtatcaacgcagagtacacgtcaaccnnnnnnnntttttttttttttttttttttttttttttt

SEQ ID NO: 82 - DNA, 72 bp, Artificial Sequence
  Description of Artificial Sequence Synthetic oligonucleotide
  modified_base (35)..(42): a, c, t, g, unknown or other
aagcagtggtatcaacgcagagtacaccttaaggnnnnnnnntttttttttttttttttttttttttttttt

SEQ ID NO: 83 - DNA, 72 bp, Artificial Sequence
  Description of Artificial Sequence Synthetic oligonucleotide
  modified_base (35)..(42): a, c, t, g, unknown or other
aagcagtggtatcaacgcagagtaccggtctacannnnnnnntttttttttttttttttttttttttttttt

SEQ ID NO: 84 - DNA, 72 bp, Artificial Sequence
  Description of Artificial Sequence Synthetic oligonucleotide
  modified_base (35)..(42): a, c, t, g, unknown or other
aagcagtggtatcaacgcagagtaccttcttaccnnnnnnnntttttttttttttttttttttttttttttt

SEQ ID NO: 85 - DNA, 72 bp, Artificial Sequence
  Description of Artificial Sequence Synthetic oligonucleotide
  modified_base (35)..(42): a, c, t, g, unknown or other
aagcagtggtatcaacgcagagtacatgctgttcnnnnnnnntttttttttttttttttttttttttttttt

SEQ ID NO: 86 - DNA, 72 bp, Artificial Sequence
  Description of Artificial Sequence Synthetic oligonucleotide
  modified_base (35)..(42): a, c, t, g, unknown or other
aagcagtggtatcaacgcagagtactgctacatgnnnnnnnntttttttttttttttttttttttttttttt

SEQ ID NO: 87 - DNA, 72 bp, Artificial Sequence
  Description of Artificial Sequence Synthetic oligonucleotide
  modified_base (35)..(42): a, c, t, g, unknown or other
aagcagtggtatcaacgcagagtacagaaccgctnnnnnnnntttttttttttttttttttttttttttttt

SEQ ID NO: 88 - DNA, 72 bp, Artificial Sequence
  Description of Artificial Sequence Synthetic oligonucleotide
  modified_base (35)..(42): a, c, t, g, unknown or other
aagcagtggtatcaacgcagagtacttgtcagtcnnnnnnnntttttttttttttttttttttttttttttt

SEQ ID NO: 89 - DNA, 72 bp, Artificial Sequence
  Description of Artificial Sequence Synthetic oligonucleotide
  modified_base (35)..(42): a, c, t, g, unknown or other
aagcagtggtatcaacgcagagtacatccaagccnnnnnnnntttttttttttttttttttttttttttttt

SEQ ID NO: 90 - DNA, 72 bp, Artificial Sequence
  Description of Artificial Sequence Synthetic oligonucleotide
  modified_base (35)..(42): a, c, t, g, unknown or other
aagcagtggtatcaacgcagagtacctagtcactnnnnnnnntttttttttttttttttttttttttttttt

SEQ ID NO: 91 - DNA, 72 bp, Artificial Sequence
  Description of Artificial Sequence Synthetic oligonucleotide
  modified_base (35)..(42): a, c, t, g, unknown or other
aagcagtggtatcaacgcagagtaccagtggcttnnnnnnnntttttttttttttttttttttttttttttt

SEQ ID NO: 92 - DNA, 72 bp, Artificial Sequence
  Description of Artificial Sequence Synthetic oligonucleotide
  modified_base (35)..(42): a, c, t, g, unknown or other
aagcagtggtatcaacgcagagtactggcacactnnnnnnnntttttttttttttttttttttttttttttt

SEQ ID NO: 93 - DNA, 72 bp, Artificial Sequence
  Description of Artificial Sequence Synthetic oligonucleotide
  modified_base (35)..(42): a, c, t, g, unknown or other
aagcagtggtatcaacgcagagtaccggtaagttnnnnnnnntttttttttttttttttttttttttttttt

SEQ ID NO: 94 - DNA, 72 bp, Artificial Sequence
  Description of Artificial Sequence Synthetic oligonucleotide
  modified_base (35)..(42): a, c, t, g, unknown or other
aagcagtggtatcaacgcagagtacggttgattcnnnnnnnntttttttttttttttttttttttttttttt

SEQ ID NO: 95 - DNA, 72 bp, Artificial Sequence
  Description of Artificial Sequence Synthetic oligonucleotide
  modified_base (35)..(42): a, c, t, g, unknown or other
aagcagtggtatcaacgcagagtaccatctactgnnnnnnnntttttttttttttttttttttttttttttt

SEQ ID NO: 96 - DNA, 72 bp, Artificial Sequence
  Description of Artificial Sequence Synthetic oligonucleotide
  modified_base (35)..(42): a, c, t, g, unknown or other
aagcagtggtatcaacgcagagtacctagtggagnnnnnnnntttttttttttttttttttttttttttttt

SEQ ID NO: 97 - DNA, 72 bp, Artificial Sequence
  Description of Artificial Sequence Synthetic oligonucleotide
  modified_base (35)..(42): a, c, t, g, unknown or other
aagcagtggtatcaacgcagagtactcactctccnnnnnnnntttttttttttttttttttttttttttttt

SEQ ID NO: 98 - DNA, 72 bp, Artificial Sequence
  Description of Artificial Sequence Synthetic oligonucleotide
  modified_base (35)..(42): a, c, t, g, unknown or other
aagcagtggtatcaacgcagagtacccacaagacnnnnnnnntttttttttttttttttttttttttttttt

SEQ ID NO: 99 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnaggccttaagtactctgcgttgataccactgctt

SEQ ID NO: 100 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnacatgtcctgtactctgcgttgataccactgctt

SEQ ID NO: 101 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnaagcctggtgtactctgcgttgataccactgctt

SEQ ID NO: 102 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnncgattcctggtactctgcgttgataccactgctt

SEQ ID NO: 103 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnncgaaggatcgtactctgcgttgataccactgctt

SEQ ID NO: 104 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnatatgacgggtactctgcgttgataccactgctt

SEQ ID NO: 105 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnttgtggactgtactctgcgttgataccactgctt

SEQ ID NO: 106 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnaggctgatggtactctgcgttgataccactgctt

SEQ ID NO: 107 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngccatagaagtactctgcgttgataccactgctt

SEQ ID NO: 108 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnncttacgagggtactctgcgttgataccactgctt

SEQ ID NO: 109 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngaattgcgagtactctgcgttgataccactgctt

SEQ ID NO: 110 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnntgcaccaaggtactctgcgttgataccactgctt

SEQ ID NO: 111 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngaagcttctgtactctgcgttgataccactgctt

SEQ ID NO: 112 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnncctccaacagtactctgcgttgataccactgctt

SEQ ID NO: 113 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnncgaatgtgggtactctgcgttgataccactgctt

SEQ ID NO: 114 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnncgaccattggtactctgcgttgataccactgctt

SEQ ID NO: 115 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnaccgcatatgtactctgcgttgataccactgctt

SEQ ID NO: 116 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnctgttacaggtactctgcgttgataccactgctt

SEQ ID NO: 117 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnncttgaggaagtactctgcgttgataccactgctt

SEQ ID NO: 118 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnaccaacttggtactctgcgttgataccactgctt

SEQ ID NO: 119 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnacctcacgagtactctgcgttgataccactgctt

SEQ ID NO: 120 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnttccgaagtgtactctgcgttgataccactgctt

SEQ ID NO: 121 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnacaggcagtgtactctgcgttgataccactgctt

SEQ ID NO: 122 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnncgagacttcgtactctgcgttgataccactgctt

SEQ ID NO: 123 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnattccaggagtactctgcgttgataccactgctt

SEQ ID NO: 124 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnccgaagagagtactctgcgttgataccactgctt

SEQ ID NO: 125 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnntccatggctgtactctgcgttgataccactgctt

SEQ ID NO: 126 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnacaacagaggtactctgcgttgataccactgctt

SEQ ID NO: 127 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnatgtgtagcgtactctgcgttgataccactgctt

SEQ ID NO: 128 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnatggatacggtactctgcgttgataccactgctt

SEQ ID NO: 129 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnacaaggacagtactctgcgttgataccactgctt

SEQ ID NO: 130 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnntcgtgccaagtactctgcgttgataccactgctt

SEQ ID NO: 131 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnaagttcgccgtactctgcgttgataccactgctt

SEQ ID NO: 132 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnggaacatacgtactctgcgttgataccactgctt

SEQ ID NO: 133 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngtgtcctaagtactctgcgttgataccactgctt

SEQ ID NO: 134 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnaaggtccgagtactctgcgttgataccactgctt

SEQ ID NO: 135 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnacaagatgcgtactctgcgttgataccactgctt

SEQ ID NO: 136 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnactcctaaggtactctgcgttgataccactgctt

SEQ ID NO: 137 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngtagctgaggtactctgcgttgataccactgctt

SEQ ID NO: 138 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnaatacctgcgtactctgcgttgataccactgctt

SEQ ID NO: 139 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngcttacgcagtactctgcgttgataccactgctt

SEQ ID NO: 140 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngagaggaatgtactctgcgttgataccactgctt

SEQ ID NO: 141 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngacgttccagtactctgcgttgataccactgctt

SEQ ID NO: 142 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnaaggaatccgtactctgcgttgataccactgctt

SEQ ID NO: 143 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnaaggcgtaggtactctgcgttgataccactgctt

SEQ ID NO: 144 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnaacgcctcagtactctgcgttgataccactgctt

SEQ ID NO: 145 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngtgcgtctagtactctgcgttgataccactgctt

SEQ ID NO: 146 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnttcggaccagtactctgcgttgataccactgctt

SEQ ID NO: 147 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngatggtgaagtactctgcgttgataccactgctt

SEQ ID NO: 148 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngttatcgacgtactctgcgttgataccactgctt

SEQ ID NO: 149 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnccatctcaggtactctgcgttgataccactgctt

SEQ ID NO: 150 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngcaggctaagtactctgcgttgataccactgctt

SEQ ID NO: 151 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngtacaactggtactctgcgttgataccactgctt

SEQ ID NO: 152 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnttcagttgggtactctgcgttgataccactgctt

SEQ ID NO: 153 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnattggtcaggtactctgcgttgataccactgctt

SEQ ID NO: 154 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnncagctatcggtactctgcgttgataccactgctt

SEQ ID NO: 155 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnctccttatcgtactctgcgttgataccactgctt

SEQ ID NO: 156 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnntctcgctgtgtactctgcgttgataccactgctt

SEQ ID NO: 157 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngcaatgagtgtactctgcgttgataccactgctt

SEQ ID NO: 158 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnncgcactcttgtactctgcgttgataccactgctt

SEQ ID NO: 159 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngaacggttagtactctgcgttgataccactgctt

SEQ ID NO: 160 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngcttgtaacgtactctgcgttgataccactgctt

SEQ ID NO: 161 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnaatcgcttggtactctgcgttgataccactgctt

SEQ ID NO: 162 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnggcaattccgtactctgcgttgataccactgctt

SEQ ID NO: 163 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnaagacgatcgtactctgcgttgataccactgctt

SEQ ID NO: 164 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnncataggctagtactctgcgttgataccactgctt

SEQ ID NO: 165 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnagtccgtgtgtactctgcgttgataccactgctt

SEQ ID NO: 166 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngactctgtagtactctgcgttgataccactgctt

SEQ ID NO: 167 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnaacctcgaagtactctgcgttgataccactgctt

SEQ ID NO: 168 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnatggccaatgtactctgcgttgataccactgctt

SEQ ID NO: 169 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnntgcgagatggtactctgcgttgataccactgctt

SEQ ID NO: 170 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnacaattcgggtactctgcgttgataccactgctt

SEQ ID NO: 171 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnaacacaagggtactctgcgttgataccactgctt

SEQ ID NO: 172 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnataatggccgtactctgcgttgataccactgctt

SEQ ID NO: 173 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnncctagtgtggtactctgcgttgataccactgctt

SEQ ID NO: 174 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnncgacaaccagtactctgcgttgataccactgctt

SEQ ID NO: 175 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnncacatggtggtactctgcgttgataccactgctt

SEQ ID NO: 176 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnatccaagccgtactctgcgttgataccactgctt

SEQ ID NO: 177 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnggttgacgtgtactctgcgttgataccactgctt

SEQ ID NO: 178 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnccttaaggtgtactctgcgttgataccactgctt

SEQ ID NO: 179 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnntgtagaccggtactctgcgttgataccactgctt

SEQ ID NO: 180 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnggtaagaaggtactctgcgttgataccactgctt

SEQ ID NO: 181 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngaacagcatgtactctgcgttgataccactgctt

SEQ ID NO: 182 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnncatgtagcagtactctgcgttgataccactgctt

SEQ ID NO: 183 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnagcggttctgtactctgcgttgataccactgctt

SEQ ID NO: 184 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngactgacaagtactctgcgttgataccactgctt

SEQ ID NO: 185 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnggcttggatgtactctgcgttgataccactgctt

SEQ ID NO: 186 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnagtgactaggtactctgcgttgataccactgctt

SEQ ID NO: 187 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnaagccactggtactctgcgttgataccactgctt

SEQ ID NO: 188 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnagtgtgccagtactctgcgttgataccactgctt

SEQ ID NO: 189 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnaacttaccggtactctgcgttgataccactgctt

SEQ ID NO: 190 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngaatcaaccgtactctgcgttgataccactgctt

SEQ ID NO: 191 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnncagtagatggtactctgcgttgataccactgctt

SEQ ID NO: 192 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnctccactaggtactctgcgttgataccactgctt

SEQ ID NO: 193 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnnggagagtgagtactctgcgttgataccactgctt

SEQ ID NO: 194 - DNA, 74 bp, Artificial Sequence
  Description of Artificial Sequence Synthetic oligonucleotide
  modified_base (1)..(1): a, c, t, or g
  modified_base (33)..(40): a, c, t, g, unknown or other
nbaaaaaaaaaaaaaaaaaaaaaaaaaaaaaannnnnnnngtcttgtgggtactctgcgttgataccactgctt

SEQ ID NO: 195 - DNA, 23 bp, Artificial Sequence
  Description of Artificial Sequence Synthetic primer
aagcagtggtatcaacgcagagt

SEQ ID NO: 196 - DNA, 32 bp, Artificial Sequence
  Description of Artificial Sequence Synthetic primer
  modified_base (1)..(1): a, c, t, or g
nbaaaaaaaaaaaaaaaaaaaaaaaaaaaaaa

SEQ ID NO: 197 - DNA, 80 bp, Artificial Sequence
  Description of Artificial Sequence Synthetic oligonucleotide
  modified_base (42)..(49): a, c, t, g, unknown or other
tttttttaagcagtggtatcaacgcagagtacttgtggtagnnnnnnnnvtttttttttttttttttttttttttttttt

(19) United States
(12) Patent Application Publication
(10) Pub. No.: US 2022/0235406 A1
(43) Pub. Date: Jul. 28, 2022

(54) METHODS AND COMPOSITIONS FOR MULTIPLE-PARAMETER SINGLE-CELL ANALYSIS USING SPECTRALLY ENCODED MICROBEADS

(71) Applicants: Chan Zuckerberg Biohub, Inc., San Francisco, CA (US); The Board of Trustees of the Leland Stanford Junior University, Stanford, CA (US)

(72) Inventors: Kara Brower, Stanford, CA (US); Polly M. Fordyce, Stanford, CA (US); Alexandra Sockell, Stanford, CA (US); Adam K. White, Stanford, CA (US); Yinnian Feng, Stanford, CA (US)

(21) Appl. No.: 17/613,892

(22) PCT Filed: May 27, 2020
(86) PCT No.: PCT/US2020/034680

§ 371 Date: Nov. 23, 2021

Related U.S. Application Data
(60) Provisional application No. 62/853,627, filed on May 28, 2019.

(51) Int. Cl. C12Q 1/6841; G01N 15/14

(52) U.S. Cl. CPC C12Q 1/6841; G01N 15/1463; G01N 2015/1497; G01N 15/1484; G01N 15/1475

(57) ABSTRACT

The invention provides compositions and methods for associating data from phenotypic analysis of a single cell with sequencing data from the single cell using spectrally-encoded microbeads.

### CROSS-REFERENCE TO RELATED APPLICATIONS

This application claims priority benefit of U.S. Provisional Application No. 62/853,627, filed May 28, 2019, which is incorporated by reference for all purposes.

### STATEMENT AS TO RIGHTS TO INVENTIONS MADE UNDER FEDERALLY SPONSORED RESEARCH AND DEVELOPMENT

This invention was made with Government support under contract GM123641 awarded by the National Institutes of Health. The Government has certain rights in the invention.

### BACKGROUND OF THE INVENTION

Recent developments in single cell analysis have provided the ability to evaluate phenotypes, profile transcript products and analyze genomes of individual cells. Microfluidic single cell transcriptomics (i.e., RNA expression transcript profile) techniques (e.g., DropSeq, InDrop, CytoSeq) employing microbeads as solid-phase supports for capture and processing of cellular RNA have been widely adopted due to their throughput, ease-of-use, and commercial standardization (e.g., Becton Dickinson Rhapsody™; 10× Genomics Gemcode™ Platform). These assays employ sequence-based barcodes, often referred to as unique cellular identifiers (UCIs), which provide trackable identifiers for all transcriptomic reads from a single cell when captured on a microbead, thus allowing transcripts from all cells to be sequenced simultaneously in a single massively parallel sequencing pool for the entire population without losing the ability to trace the single cell from which transcripts originated. Such techniques often rely on a randomized, ‘split and pool’ synthesis approach to generate their unique cell identifier per bead. However, these bead-based assays are not capable of analyzing single cells for more than one parameter (e.g., analyzing more than one class of biomolecular parameters and/or cellular phenotype) because they lack an ability to link the transcriptomic single cell barcode identifier sequences with a secondary parameter that is analyzed, particularly for parameters that are not easily integrated with massively parallel sequencing.

Methods to analyze single cells are important for evaluating cell-to-cell variability in biological systems, where the characteristics of minority cell populations can be lost in bulk, population-averaged measurements. Quantifying the abundance, organization, and differential expression of biomolecules (e.g., genomic DNA, chromatin structure and organization, RNA, and proteins) in single cells has proven important in determining the mechanisms that drive complex biological processes and disease states having heterogeneous cell populations, such as intra-tumor growth, proliferation and recurrence, immune regulation, and early organism development. Analysis of individual cells has traditionally been limited due to challenges associated with small amounts of starting material, such as cost, and labor required to examine large numbers of cells, as well as difficulties in adapting bulk-methods to small volumes.

Cellular phenotyping methods that classify, quantify, and monitor the function, behaviors, and responses of individual cells, either isolated or in their tissue context, have been equally important for elucidating biological mechanisms of action in development, regulation, drug response, or disease. As noted above, however, limited technologies currently exist that allow for matching sequence-based biomolecule quantification using sequencing (e.g., gene expression via RNA-Seq) to phenotypic characterization (e.g., dynamic cellular imaging) of a particular single cell. The few attempts of a multi-parameter single cell analysis approach involving functional or phenotypic analysis of single cells in tandem with a sequencing-based measurement have been throughput-limited and/or require high per-cell run-costs (as is the case with FACS+plate-based Next Generation Sequencing (NGS) methods). This gap is currently filled by computational methods which attempt to extrapolate a relationship between statistical clusters of cells displaying certain phenotypes with a separate measurement of single cell genotypes in samples of high heterogeneity.

### BRIEF SUMMARY OF CERTAIN ASPECTS OF THE INVENTION

The methods and compositions described in the present disclosure use a markedly different approach for single cell analysis compared to the previous randomized pool-directed synthesis techniques. Unlike ‘split and pool’ synthesis approaches of many previous techniques that are used to generate their unique cell identifier per bead (which is subsequently linked to the transcriptomic reads), an assay performed in accordance with the present invention uses a 1:1 linkage of a spectral signature (color) and an identifier sequence attached to a bead, as further described below. This additional layer of linkage provides the ability to determine which single cell in a population being analyzed has a given characteristic and moreover, provides the ability to link an additional characteristic of interest to the same single cell.

As described herein, each microbead has a plurality of capture oligonucleotides immobilized to it for hybridization to capture one or more nucleic acid populations of interest. The capture oligonucleotides for each bead comprise two domains: (i) a spectral signature identifier domain that is shared by the plurality of capture oligonucleotides and has a sequence distinct for each spectral signature encoded by a bead, i.e., the capture oligonucleotides immobilized to one bead can be distinguished by the spectral identifier sequence of the capture oligonucleotides, and thus can be linked to the spectral signature of a bead; and (ii) a capture domain that comprises a nucleic acid sequence that is substantially complementary to a target sequence of a desired population of polynucleotides, e.g., mRNA or genomic DNA, in a single cell. A spectral signature is introduced into each microbead employed in the bead-based assay. The spectral signature is created by ratiometric incorporation of lanthanide nanophosphors embedded within the bead. A 1:1 linkage is created with the spectral code (i.e., color) of the bead) and the spectral signature identifier sequence. Use of such beads provides the ability to couple spatial (e.g., microwell or other compartment positional information), sequence (bead-based cell identification sequence) and optical/spectral—(i.e., microbead spectral signature or ‘code’) information to precisely track data generated from a single cell in tandem sequencing-based and phenotypic analysis, i.e., data from the phenotypic and sequencing-based analyses can be traced to the single cell from which the data originated.

Thus, in one aspect, the disclosure describes a single cell analysis technology for making single cell (SC) tandem sequence-based nucleic acid measurements and phenotypic measurements at population scale (e.g., 100-1000 cells) by harnessing a spectrally encoded bead as the coding link between single cell parameters that are measured. This method thereby provides the ability to analyze each cell over multiple parameters (e.g., gene expression via RNA-Seq linked to cellular morphology and/or intracellular protein dynamics via imaging) in tandem, without sophisticated statistical methods to couple separate single cell population measurements and without difficult physical separation methods to evaluate the multi-parameter readouts. Detailed herein is an integrated platform (see, e.g., the illustrative embodiments depicted in FIG. 1) that employs spatial—(e.g., microwells), spectral—(e.g., lanthanide-encoded microbeads), and sequence—(oligonucleotide spectral signature identifier sequence) encoding to perform sensitive same-cell multi-parameter analysis that precisely links phenotype (e.g., cell imaging) and biomolecular data quantified via massively parallel sequencing (e.g., gene expression) for a large number of cells, e.g., 100-1000 cells, or greater, in parallel.

In one aspect, provided herein is a method of analyzing multiple parameters in a single cell, the method comprising:

(i) generating an arrayed configuration comprising a plurality of single-cell analysis compartments, wherein each single cell analysis compartment comprises:
(a) a single cell or nucleus, and
(b) a single microbead having a lanthanide spectral signature, and immobilized on said microbead, a plurality of capture oligonucleotides that comprises a first and a second domain, where the first domain comprises a nucleic acid spectral signature identifier sequence specific for the lanthanide spectral signature of the single microbead, and the second domain comprises a nucleic acid sequence that is substantially complementary to a sequence of one or more target polynucleotides from the cell or nucleus;
wherein the lanthanide spectral signature of each microbead present in each single-cell analysis compartment of the plurality of single-cell analysis compartments is distinguishable from the lanthanide spectral signature of the microbeads in the other single-cell analysis compartments, and the capture oligonucleotide and microbead in each single-cell analysis compartment are unambiguously paired;
(ii) imaging the arrayed configuration to determine spatial position of the single cell or nucleus and the microbead contained in each single-cell analysis compartment;
(iii) for at least some of the compartments, and preferably all of the compartments that contain a single bead, determining the spectral signature of the microbead contained therein;
(iv) analyzing a first parameter of the cell or nucleus contained therein, wherein analyzing comprises detecting a signal from the single-cell analysis compartment and/or recording an image of the single cells contained in at least a portion of the single cell analysis compartments;
(v) for at least some of the single-cell analysis compartments in (iii), maintaining the single-cell analysis compartments under conditions in which the capture oligonucleotides hybridize to said one or more target polynucleotides;
(vi) analyzing a second parameter of the single cell wherein the second parameter comprises determining the sequence at least a portion of one or more target polynucleotides; and
(vii) determining the sequence of at least a portion of a capture oligonucleotide, where the sequence is sufficient to unambiguously pair the capture oligonucleotide with the microbead in the single-cell analysis compartment. In some embodiments, the contents of multiple single cell analysis compartments are combined before claim step (vi). In some embodiments, step (iv) comprises performing an image-based assay. In some embodiments steps (ii) and (iii) are performed at the same time. In some embodiments, step (ii) and step (iii) are performed after step (v). In some embodiments the array is an ordered array. In some embodiments, the signal detected in (iv) is a chemiluminescent signal or a fluorescent signal. In some embodiments the single cell analysis compartments further comprise reagents to assay the first parameter. In some embodiments, first parameter is expression of a secreted protein or marker expressed on the cell surface. In such embodiments, the single cell analysis compartments may further contain one or more antibodies to detect the marker expressed on the cell surface or the secreted protein. In some embodiments, reagents for the assay comprise intracellular dyes or stains. In some embodiments, the first parameter assayed is lipid or carbohydrate content of the single cell. In some embodiments, the single cell analysis compartments comprise a single cell and the first parameter is cell morphology or cell motility, e.g., imaged by phase contrast, brightfield, or differential interference contrast (DIC) microscopy; or in some embodiments, tracked or imaged using fluorescent or luminescent dyes associated with lipid membrane, organelles, nuclear envelops, and/or cellular protein lipid, carbohydrate or cytoskeletal content. In some embodiments, step (ii) is performed two or more times over desired intervals of time. In some embodiments, the second parameter is expression of the population of polynucleotides of interest. In some embodiments, the method further comprises a step of lysing the single cell or nucleus in each single cell-analysis compartment performed after step (ii). In some embodiments, the method further comprises a step of collecting the microbeads bound to the population of polynucleotides of interest from the single-cell compartments and/or a step of processing the polynucleotides bound to the microbeads to generate reaction products. Processing may include, for example performing an amplification reaction, such as an RT-PCR reaction. In some embodiments, the capture molecule is a capture oligonucleotide that comprises a nucleic acid sequence that hybridizes to the target polynucleotides of interest, e.g., genomic DNA, chromatin, or RNA, e.g., mRNA. In some embodiments, the nucleic acids captured by the beads are further processed for massively parallel sequencing. For example, in some embodiments, a reverse transcriptase reaction is employed so that cDNA is attached to the bead surface and followed by an amplification reactions, e.g., PCR, to generate amplicons that can then be pooled for sequencing. In some embodiments, the capture oligonucleotide further comprises a unique molecular identifier nucleic acid sequence and optionally, the method may further comprise a step of collecting the microbeads from the single-cell analysis compartments following step (v). In some embodiments, the method comprises processing mRNA captured by the capture oligonucleotide for RNA-Seq analysis. Thus, for example, in some embodiments, the processing step comprises a reverse transcriptase reaction to produces cDNA and subsequent amplification of the cDNA. In some embodiments, the population of biomarkers comprises genomic DNA and the method further comprises a step of processing the genomic DNA for genotyping, haplotype analysis, or methylation profiling.

In a further aspect, provided herein is a method of analyzing multiple parameters in a single cell, the method comprising:

(i) generating an arrayed configuration comprising a plurality of single-cell analysis compartments, wherein each single-cell analysis compartment contained in the plurality of single-cell analysis compartments comprises

(a) a single cell, and

(b) a single microbead having a lanthanide spectral signature, and immobilized on said microbead, a plurality of capture oligonucleotides comprising a first and a second domain, where the first domain comprises a nucleic acid sequence specific to the lanthanide spectral signature and the second domain comprises a nucleic acid sequence that is substantially complementary to a target mRNA population from the cell;

wherein the lanthanide spectral signature of a microbead present in one single-cell analysis compartment is distinguishable from the lanthanide spectral signature of the microbeads in the other single cell analysis compartments, and the capture oligonucleotide and spectral signature of the microbead in each single-cell analysis compartment are unambiguously paired;

(ii) imaging the arrayed configuration to determine spatial position of the cell and the microbead contained in each single-cell analysis compartment;
(iii) for at least some compartments, determining the spectral signatures of each microbead in each single cell analysis compartment;
(iv) analyzing a first parameter of the cell contained there, wherein analyzing comprises detecting a signal from the single-cell analysis compartment; and/or recording a microscope image of the single cell contained in at least a portion of the single cell analysis compartments;
(v) maintaining at least some of the single cell analysis compartments in (iii) in which the capture oligonucleotide captures the target mRNA population; and
(vi) recovering the microbeads hybridized to the target mRNA population;
(vii) processing the target mRNA population for RNA-Seq, e.g., by performing a reverse transcriptase reaction with the RNA hybridized to the capture domains present on the microbeads followed by a PCR reaction to generate reaction products that are not physically coupled to the beads to pool for massively parallel analysis; and
(vii) performing RNA-Seq analysis. In some embodiments, the second domain comprises a poly(T) tract. In some embodiments, each single cell analysis compartment further comprises an assay reagent for analyzing the first parameter, e.g., antibodies to assess protein expression. In some embodiments, analyzing the first parameter comprises analyzing: expression of one or more proteins, such as secreted proteins, proteins expressed on the surface of cells, and or intracellular proteins; or analyzing cell motility, cellular lipid content, cellular carbohydrate content, cell morphology, cytoskeleton structure, or functional response to selective perturbation or differential drug treatment. In some embodiments, step (ii) is performed two or more times over a desired interval of time. In some embodiments, step (ii) and (iii) are performed at the same time. In some embodiments, the capture oligonucleotide further comprises a unique molecular identifier nucleic acid sequence. Also provided herein are methods that further comprise a step of collecting the microbeads from the single-cell analysis compartments following step (v).

In some embodiments of the methods of the present invention, the arrayed configuration is generated in a microwell device comprising an array of microwells. In illustrative embodiments, the microwell device comprises a clamping slide and the array of microwells. Such a microwell device may further comprises an electrode slide. In some embodiments a microwell device employed in the methods of the present invention is a microfluidic device. For example, in some embodiments the microwell size is about 50 to about 1,000 microns. Often, the microwell size is less than about 500 microns. In some embodiments, the arrayed configuration is generated by droplets deposited in individual wells of a microwell device or deposited in a defined pattern on a slide.

A single cell in the single cell analysis compartments may be a prokaryotic cell or eukaryotic cell. In some embodiments, the single cell or nucleic in the single cell analysis compartment is a mammalian cell, e.g., a cancer cell, lymphocyte, or any other cell type; or a yeast cell.

In a further aspect, provided herein is an arrayed configuration comprising a plurality of single-cell analysis compartments, wherein each single-cell analysis compartment contained in the plurality of single-cell analysis compartments comprises:

(a) a single cell or nucleus, and

(b) a single microbead having a lanthanide spectral signature, and immobilized on said microbead, a plurality of capture oligonucleotides that comprises a first and a second domain, where the first domain comprises a nucleic acid sequence specific to the microbead and the second domain comprises a nucleic acid sequence that is substantially complementary to one or more target polynucleotides from the cell or nucleus;

wherein the lanthanide spectral signature of a microbead present in one single-cell analysis compartment is distinguishable from the lanthanide spectral signature of the microbeads in the other single cell analysis compartments, and the capture oligonucleotide and lanthanide spectral signature of each microbead in each single-cell analysis compartment are unambiguously paired.

In some embodiments, each single-cell analysis compartment of the plurality of single-cell analysis compartments comprises assay reagents. In some embodiments, the capture oligonucleotide further comprises a unique molecular identifier sequence. In some embodiments, the arrayed configuration comprises a microwell device comprising an array of microwells. In some embodiments, the microwell device comprises a clamping slide and the array of microwells. In some embodiments, the microwell device further comprises an electrode slide. In some embodiments, the arrayed configuration comprises at least 100 different lanthanide spectral signatures. In some embodiments, the arrayed configuration comprises at least 1000 different lanthanide spectral signatures.

### BRIEF DESCRIPTION OF THE DRAWINGS

FIG. 1, panels A-C depict an illustrative and non-limiting embodiment of a spectrally encoded microbead-based platform/device for multi-parameter single cell analysis as described in the present disclosure.

FIG. 2, panels A-D provide an illustration of microbead/capture oligonucleotide configuration and analysis of sequencing data.

FIG. 3, panels A-E depict an illustrative embodiment comprising an image-based phenotypic assay and association with a single cell based on spectral code.

### DETAILED DESCRIPTION

### Terminology

A “parameter,” in the context of the present disclosure, refers to a characteristic of a cell or cell component (e.g., transcriptome) of interest. A “parameter” is determined by an “assay.” Exemplary assays include, but are not limited to, binding assays, immunoassays, amplification assays, sequencing assays, hybridization assays, assay to image a cell, and the like. As used herein with respect to a multi-parameter analysis, a “phenotypic” assay refers to an assay that detects a signal from a cell in at least a portion of single-cell analysis compartments and/or comprises recording a microscopic image of single cells contained in at least a portion of the single cell analysis compartments. Such a phenotypic assay thus measures a cellular characteristics other than nucleic acid sequences. A “sequence-based” assay refers to analysis of nucleic acid sequences in a single cell, or nucleus, that can be performed in a massively parallel sequencing platform to analyze data obtained from a plurality of single cells, e.g., from 100-1,000.

An “image-based” parameter as used herein refers to a parameter that comprises a step in which single cells are visualized and imaged, e.g., by brightfield, phase, or DIC microscopy, confocal imaging, z-stack/volumetric imaging, and the like. Such an assay may also employ additional reagents, e.g., fluorescent or luminescent dyes or other labels, that generate a signal.

The term “unambiguously paired” in the context of a spectral signature of a microbead and the spectral signature identifier sequence domain specific to the color (spectral signature) of the microbead refers to the association of one spectral signature identifier domain with one spectral signature, thereby providing the ability to identify the bead having that color in a population of beads based on the identifier domain sequence. As described herein, the spectral signature identifier domain is shared among the individual capture oligonucleotides present in a plurality of capture oligonucleotides immobilized to a bead; and can be distinguished from the spectral signature identifier domains of other beads in the assay.

A “polynucleotide” or “nucleic acid” includes any form of RNA or DNA, including, for example, genomic DNA; complementary DNA (cDNA); and DNA molecules produced synthetically or by amplification. Polynucleotides may include chimeric molecules and nucleic acids comprising non-standard bases (e.g., inosine) or nucleotide analogs. For example, an oligonucleotide may contain naturally occurring nucleotides and/or analogs thereof. Polynucleotides may be single-stranded or double-stranded.

A “target polynucleotide” or “target nucleic acid” is a polynucleotide that comprises a target sequence. In a double-stranded target polynucleotide the target sequence is on one strand and the complement of the target sequence is on the other strand. A “target RNA” is an RNA that comprises a target sequence.

The term “substantially complementary” refers to a sequence that is not perfectly complementary to its target sequence, but can hybridize selectively to the desired target sequences. Such a sequence may have one or more “mismatches”, i.e., one or more positions in the sequence in which the nucleotide in the capture domain and the nucleotide in the target nucleic acid with which it is aligned are not complementary. Selectivity of hybridization exists when hybridization occurs that is more selective than total lack of specificity. Typically, selective hybridization will occur when there is at least about 55% identity over a stretch of at least 14-25 nucleotides, preferably at least 65%, more preferably at least 75%, and most preferably at least 90%. See M. Kanehisa, Nucleic Acids Res. 12:203 (1984).

The terms “anneal”, “hybridize” or “bind,” in reference to two polynucleotide sequences, segments or strands, are used interchangeably and have the usual meaning in the art. Two complementary sequences (e.g., DNA and/or RNA) anneal or hybridize by forming hydrogen bonds with complementary bases to produce a double-stranded polynucleotide or a double-stranded region of a polynucleotide.

As used herein the term “lanthanide spectral signature” refers to the combined luminescent signals in the range of 350-850 nm emitted from lanthanide nanoparticles contained in a single microbead upon excitation with an appropriate wavelength of light, e.g., UV light (e.g., 292 nm for excitation of downconverting lanthanides) or IR light (e.g., 980 nm for excitation of upconverting lanthanides). The luminescence intensity at a characteristic wavelength or wavelengths (e.g., 620 nm, 630 nm, or 650 nm) for a particular lanthanide (e.g., Eu) indicates the presence and quantity of the particular lanthanide in the source (e.g., a microbead) from which the spectral signature originates. “Lanthanide” refers to elements 57-71 of the periodic table, namely lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu). A spectral signature is determined by deriving the relative proportions of each lanthanide species in each bead from the images taken at the characteristic wavelengths for each lanthanide during emission.

As used herein, the term “microbead” refers to a particle having one or more dimensions (e.g., length, width, diameter, or circumference) of about 1000 μm or less, e.g., less than about 500 μm, 100 μm, or 10 μm. Microbeads may have a generally spherical shape or a non-spherical shape. Microbeads used in the methods of the present disclosure are characterized by a detectable spectral signature as described in more detail below. A “plurality” of microbeads refers to a population of microbeads ranging in size from a few microbeads to thousands of microbeads, or more.

The term “label,” as used herein, refers to any atom or molecule that can be used to provide a detectable and/or quantifiable signal. In some embodiments, the label can be attached, directly or indirectly, to a biomolecule. Labels include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

An “array configuration” as used herein refers to a collection of single compartments, at least a portion of which each contain a single cell and a single microbead co-compartmentalized with the single cell. An array configuration may be an “ordered array” in which the compartments are addressable and can be assigned to known locations.

A “compartment” as used herein refers to any partially or fully enclosed space that separates one single cell/microbead from another. Thus, a compartment can include microwells, droplets, micropores, microfluidic chambers, and the like.

### Overview of Assay and Devices

The present invention employs an arrayed configuration that comprises a plurality of single-cell analysis compartments, at least a portion of which contain a single biological entity, typically a cell, or an organelle from a cell such as a nucleus, and a spectrally encoded microbead that can be distinguished from the other microbeads in the other single-cell analysis compartments based on its spectral signature. The single cells can be assayed to measure more than one parameter of interest, including, but not limited to, RNA expression, haplotype or genotype profile, protein expression, cellular morphology, cellular motility, or any other measure that can be assessed by high throughput, massively parallel sequencing, image analysis, or any other phenotypic analysis, e.g., that detects a signal that labels biomolecules of interest. Sequence data obtained from high throughput sequencing of nucleic acids pooled from a population of single cells can be assigned to the single cell from which it originated via a spectral signature identifier sequence that is included in the capture oligonucleotides immobilized to the microbead that is co-compartmentalized with the single cell, i.e., the spectrally-encoded lanthanide microbeads with oligonucleotide sequences conjugated to the beads have a 1:1 correspondence between the spectral signature identifier sequence and the spectral signature of the microbead to link sequence data to phenotypic data obtained from the same single cell.

In some instances, e.g., when the arrayed configuration is generated by random loading of microbeads, the resulting configuration may contain a low percentage, e.g., less than 5%, of compartments that contain a microbead having the same encoded spectral signature as a microbead in another compartment, thus resulting in ambiguity when associating the spectral signature identification sequence with a specific microbead and hence a single cell. In these instances of redundancy, data from degenerate spectral signatures can be discarded during phenotypic analysis and sequencing analysis. However, in typical embodiments the majority of microbeads in an arrayed configuration each have a unique spectral signature that allows unambiguous mapping of sequence data to the specific microbead in a compartment and hence the single cell that shares that compartment.

Spectrally encoded microbeads (also referred to herein as “beads), e.g., 10-80 micrometers in size, containing lanthanide nanophosphors at high monodispersity (C.V.<5%) are synthesized as described in U.S. Pat. Appl. Pub. No. 2015/0192518 and as further detailed below. A large numbers of spectral codes, also referred to herein as spectral signatures, that represent a unique pattern of spectral emissions such that a particular signature can be identified with high confidence, have been generated by employing precisely tunable ratios of different species of luminescent lanthanide nanophosphors in a PEG hydrogel matrix, generating droplets, and subsequently polymerizing them into microbeads using custom microfluidics (see, e.g., U.S. Pat. No. 10,241,045 and U.S. Pat. Appl. Pub. No. 2015/0192518) or custom volumetric flow instrumentation. Due to the narrow emission spectra of lanthanides, a large number of spectral codes can be achieved, e.g., 1,102 spectral codes, larger than any commercially available encoded microbead set.

The methods may be performed in any microfluidic compartmentalization platform, including, for example, microwells, droplets, micropores and the like, so long as a single cell can localized with a single microbead having a spectral signature distinguishable from the other microbeads in an array.

In some embodiments, a microwell device is used for co-compartmentalizing a bead and a single cell. In some embodiments, a micro well device may comprises anywhere from 100 to 500,000 microwells, e.g., 1,000-10,000 microwells. In some embodiments, the microwell device comprises from 1,000 to 5,000 microwells. As an example, a microwell device may comprise 10,000 microwells at 100×100 micrometers in size with 100 micrometers in spacing. In some embodiments, the single cell analysis compartments may range in size from about 30-250 micrometers. In particular embodiments, the well (or compartment) size may be selected such that only 1 bead fits per compartment/well, which provides the ability to achieve super-Poisson statistics for bead distribution to compartments.

In typical embodiments, an array is generated in which spectral beads are distributed to individual compartments in an array (e.g., a microwell, or a droplet). For example, an array may be loaded with spectral beads using a bead solution at a concentration such that >50% of the compartments are occupied by beads. Cells are loaded at set dilution concentrations (in accordance with Poisson loading) such that a portion, e.g., about 11-20% of the compartments will contain a single cell, i.e., the probability of multi-cell loading is reduced via Poisson statistics. In ideal distributions to obtain a plurality of wells containing one cell and one bead each, nearly all wells containing both microbeads and cells will be unique in their spectral code assignment due to the Poisson loading of single cells and excess loading of the microbeads.

In some embodiments, the array is a microwell device. In some embodiments, a microwell device may be clamped after addition of any reagents employed to measure one or more desired phenotypic parameters. In some embodiments, a semi-permeable membrane is employed to accommodate the addition of lysis reagents during the assay. In further embodiments, the array may comprise a population of droplets that are distributed to addressable locations.

### Preparation of Microbeads

The microbeads of the present disclosure may include different types of lanthanide nanoparticles. The nanoparticles have at least one dimension (e.g., length, width, or circumference) ranging from 1 to 1,000 nm. A microbead may include one or more different lanthanide nanoparticles, e.g., 2 or more, 3 or more, 4 or more, 5 or more, 6 or more, 7 or more, 8 or more, 9 or more, or 10 or more, wherein each lanthanide nanoparticle has a different luminescence emission spectrum upon excitation. For example, in some embodiments, the microbeads disclosed herein may include from 1 to 10, from 2 to 10, from 3 to 10, from 4 to 10, from 5 to 10, from 6 to 10, from 7 to 10, from 8 to 10, or from 9 to 10 types of lanthanide nanoparticles, wherein each lanthanide nanoparticle has a different luminescence emission spectrum upon excitation. Signals from the combined luminescence spectra make up the spectral signature of a particular microbead, and are mapped to a unique spectral signature ‘code’ during code deconvolution.

Lanthanide spectral signatures employed in the methods of the presence disclosure include emitted light in the range of 350-850 nm (e.g., 400-800 nm), exhibiting one or more peaks associated with lanthanide luminescence. Lanthanide nanoparticle spectra are typically characterized by narrow emission bands (also referred to as “signals”) in the visible region, making one species of material easily distinguishable from another. A “specific” lanthanide spectral signature in a microbead or other material can therefore be designed based on the particular identity and relative amounts of lanthanides in the microbead. In some embodiments, each of the lanthanide spectral signatures comprises an Eu signal, a Dy signal, an Sm signal, a Ce signal, a Tb signal, a La signal, a Pr signal, an Nd signal, a Gd signal, an Ho signal, an Er signal, a Tm signal, a Yb signal, a Pm signal, an Lu signal, or a combination thereof. The microbeads of the present disclosure generally include one or more different lanthanide nanoparticles as discussed herein and one or more polymers, copolymers, or combinations thereof. In some embodiments, each microbead further comprises a crosslinked polymer, wherein the capture polynucleotides are covalently bonded to the crosslinked polymer. In some embodiments, the crosslinked polymer is a hydrogel-forming polymer (e.g., poly(ethylene glycol)) which can evenly and irreversibly entrap the lanthanide nanoparticle materials within the microbead. The lanthanide nanoparticles themselves may be coated with a polymer such as poly(acrylic acid) as described in more detail below.

In some embodiments, the nanoparticles include a lanthanide and a host lattice. Lanthanides which may be incorporated into the lanthanide nanoparticles include, for example, Ce, Pr, Nd, Sm, Eu, Gd, Tb, Dy, Ho, Er, Tm, Yb, La, combinations thereof, compounds containing Ce, Pr, Nd, Sm, Eu, Gd, Tb, Dy, Ho, Er, Tm, Yb, La and combinations thereof, and ions of Ce, Pr, Nd, Sm, Eu, Gd, Tb, Dy, Ho, Er, Tm, Yb, La and combinations thereof. Host lattices employed in the nanoparticles generally contain constituent atoms packed in a regularly ordered, repeating pattern which can accommodate the incorporate of lanthanide atoms or ions. The lattice can be crystalline, which a structure that is, e.g., triclinic, monoclinic, orthorhombic, tetragonal, trigonal, hexagonal, or cubic. A crystalline lattice may contain one or more regions, i.e., grains, with distinct crystal boundaries. The crystalline lattice may, in some instances, contain two or more crystal geometries. A number of suitable host lattices can be utilized in the lanthanide nanoparticles. For example, lanthanide dopants may be incorporated into a host lattice to provide lanthanide-doped yttrium orthovanadate (YVO₄), lanthanide-doped oxides (e.g., doped ZrO₂, doped TiO₂, doped BaTiO₃), lanthanide-doped halides (e.g., doped LaF₃), lanthanide-doped phosphates (e.g., dope LaPO₄, doped LuPO₄, or doped YbPO₄), and lanthanide-doped strontium borates (e.g., SrB₄O₇, SrB₆O₁₀, and Sr₄B₁₄O₂₅), among others.

Lanthanide nanoparticles according to the present disclosure may be prepared using methods such as those described by Xu et al. (Solid State Communications, 2004. 130:465-468), Choi et al. (Journal of Luminescence. 2010. 130:549-553), Wang et al. (Angewandte Chemie International Edition, 2008. 47:906-909), and Nguyen, et al. (2017) Advanced Optical Materials, 5(3): 1600548, the disclosures of which are incorporated by reference herein in their entirety. As a non-limiting example, one volume of aqueous lanthanide dopant solution (e.g., Sm(NO₃)₃, Dy(NO₃)₃, Eu(NO₃)₃; 0.1 M), can be combined with 10-20 volumes of an yttrium salt solution (e.g., Y(NO₃)₃, 0.1 M) and added portion-wise to an 10-100 additional volumes of osmogent solution (e.g., ˜2000 kDa PEG, 10% w/w), optionally containing a bismuth salt such as Bi(NO₃)₃. A solution of matrix material (e.g., 10-100 volumes of Na₃VO₄, 0.1 M) is added portion-wise prior to microwave heating (e.g., 180° C.) for 5-120 min. Following heating, the resulting white material can be washed and resuspended (e.g., in water with optional polyacrylic acid (1000-2000 kDa; 1-10⁰% v/v)), with our without sonication and/or filtering (e.g., through 0.45-μm PTFE filters) filters to obtain the final nanoparticles, 25-250 nm in size (e.g., 30-160 nm), as milky white solutions with concentrations ranging from about 5 mg/mL to about 500 mg/mL.

Lanthanide nanoparticles according to the present disclosure may be up-converting or down-converting lanthanide nanoparticles. Suitable up-converting lanthanide nanoparticles may include, for example, NaGdF₄: Tm; NaGdF₄: Ln; NaGdF₄Yb; NaGdF₄Er; NaGdF₄Yb, Er; NaYF₄:Er; NaYF₄:Yb; NaYF₄:Er,Yb; NaYF₄:Tm,Yb; LaF₃:Yb,Tm; LaF₃:Yb,Er; and LaF₃:Yb,Ho nanoparticles. Suitable down-converting lanthanide nanoparticles may include, for example, YVO₄:Eu; YVO₄:Dy; and YVO₄:Sm nanoparticles. It should be noted that the above referenced lanthanides may be incorporated into the nanoparticles as their respective ions. Materials may be added during preparation of the lanthanide nanoparticles to increase their UV absorption, for downconverters, or IR absorption, for upconverters. For example, in some embodiments bismuth is incorporated into the lanthanide nanoparticles to increase their UV absorption.

In some embodiments, lanthanide nanoparticles as disclosed herein may be modified (e.g., covered or coated) in a suitable material to facilitate formation of a stable colloid suspension of the lanthanide nanoparticles in a carrier fluid. Suitable materials may include materials which prevent aggregation of the lanthanide nanoparticles in the carrier fluid (e.g., H₂O) and/or facilitate maintenance of a nanoparticle form of the lanthanide nanoparticles. For example, suitable materials which may be used to cover or coat the lanthanide nanoparticles may include polyethyleneimine (PEI), polyacrylic acid (PAA), sodium citrate, or citric acid. Polyethyleneimine (PEI) may be suitable for use, e.g., as a coating material in order to make the nanophosphors more compatible with a monomer mixture. In some embodiments, the nanoparticles are coated with PAA. Advantageously, PAA can enhance the photostability of the nanophosphors in addition to facilitating stable colloid formation.

Accordingly, some embodiments of the present disclosure provide a plurality of microbeads wherein each of the microbeads comprises a plurality of lanthanide nanoparticles. In some embodiments, the lanthanide nanoparticle comprises a lanthanide-doped host lattice. In some embodiments, the host lattice is yttrium orthovanadate, lanthanum phosphate, or a combination thereof.

The microbeads of the present disclosure can contain a variety of polymers. In some embodiments, the polymers form microbeads upon polymerization via, for example, a thermal- or photo-initiated polymerization process. Such polymers include, but are not limited to, polyacrylates, polyacrylamides, polymethacrylates, polymethacrylamides, polystyrenes, polythiol-enes, polyurethanes, epoxy resins, polysaccharides (such as agarose), as well as copolymers (e.g., random copolymers or block copolymers) or combinations of two or more of the above. Suitable polymers also include polysiloxanes, polyethers (e.g., polyethylene glycol (PEG)), polyvinylpyrrolidones, vinyl ethers, vinyl acetates, polyimides, polysulfones, polyamic acids, polyamides, polycarbonates, polyesters, and copolymers or combinations of two or more of the above. The polymers may be provided in monomer form during the microbead preparation process, and these monomers may be polymerized to form the above polymers, copolymers or combinations thereof in the spectrally encoded microbeads of the present disclosure. Suitable monomers may include those which can be polymerized in situ alone or with a cross-linking agent to form a cross-linked resin.

In some embodiments, polyacrylate or polyacrylamide microbeads can be prepared using monomers which contain functionalized PEGs. The functionalized PEG can contain a polymerizable functional group on each end of the PEG chain, e.g., a PEG-diacrylate or a PEG-diacrylamide for formation of crosslinked polymers that contact the lanthanide nanoparticles (e.g., a crosslinked PEG that contacts PAA-coated lanthanide nanoparticles). Alternatively, the functionalized PEG can contain a polymerizable functional group on one end of the PEG chain and an orthogonal reactive moiety on the other end of the PEG chain. The orthogonal reactive moiety can be used for the attachment of oligonucleotides or other elements (e.g., dyes, labels, or the like). Examples of orthogonal reactive moieties include, but are not limited to, amines, carboxylates, thiols, activated esters (e.g., N-hydroxysuccinimidyl (NHS) esters, sulfo-NHS esters, and pentafluorophenyl (PFP) esters); carbodiimides; maleimides; halogenated acetamides; hydroxymethyl phosphines; aryl azides; imidoesters; isocyanates; vinyl sulfones; pyridyl disulfides; benzophenones; azides; alkynes (including linear alkynes and cycloalkynes); and tetrazines.

In some embodiments, a suitable monomer for use in preparation of the microbeads is selected from a PEG diacrylamide (PEG-DAM), a PEG monoacrylamide-monoamine (PEG-AM) and a PEG-monoacrylamide-monoBoc. Such monomers can contain any suitable branched or linear PEG. In some embodiments, the PEG is a linear polymer having a weight average molecular weight ranging from 500 g/mol to about 10,000 g/mol (e.g., about 700 g/mol, about 2000 g/mol, or about 5,000 g/mol). If necessary, number average and weight average molecular weight values can be determined by gel permeation chromatography (GPC) using polymeric standards (e.g., polystyrene or like material).

Additional monomers which may be utilized in the microbeads may include, e.g., monomers which are capable of participating in thiol-ene thiol-yne reactions, e.g., pentaerythritol tetrakis(3-mercaptopropionate) (TT); diallyl phthalate (DAP); 1,3,5,-triallyl-1,3,5-triazine-2,4,6(1H,3H,5H)-trione (TIT); 1,7-octadiyne (OY); mercaptoacetic acid (MA); allylamine (AA), pentaerythritol triallyl ether (PTE) and propargylamine (PA). These monomers find use, for example, in photo-initiated polymerization processes. For additional discussion of thiol-ene thiol-yne reactions and monomers suitable for use therein, see, e.g., Prasath et al. (Polym. Chem., 2010 1: 685-692), the disclosure of which is incorporated by reference herein.

The microbeads can be prepared using microfluidic devices as described, for example, in U.S. Pat. No. 10,241,045, U.S. Pat. Appl. Pub. No. 2015/0192518, and Nguyen, et al. (Adv Opt Mater. 2017, 5 1600548), which are incorporated herein by reference in their entirety. Preparation of the microbeads may include, for example: (i) mixing at least two fluids into a first solution, wherein each fluid comprises a polymerizable component (e.g., a polymer or monomer), a polymerization initiator, and a different lanthanide nanoparticle; (ii) forming droplets from the solution; and (iii) subjecting the droplets to polymerization conditions, thereby producing a set of polymeric microbeads embedded with at least two different lanthanide nanoparticles. In some embodiments, the relative concentrations of the lanthanide nanoparticles are substantially equal (i.e., not significantly different) among the polymeric microbeads in the set. Additional sets of microbeads can be prepared by mixing the fluids into additional solutions, wherein the concentration of at least one of the lanthanide nanoparticles in the addition solutions is different than the concentrations of the nanoparticles in (i) above, and conducting the droplet-forming steps and polymerization steps as set forth above.

The lanthanide nanoparticle contained in each fluid may be present at a concentration of from about 1 mg/mL to about 250 mg/mL, e.g., from about 5 mg/mL to about 250 mg/mL, from about 10 mg/mL to about 250 mg/mL, from about 20 mg/mL to about 250 mg/mL, from about 30 mg/mL to about 250 mg/mL, from about 40 mg/mL to about 250 mg/mL, from about 50 mg/mL to about 250 mg/mL, from about 60 mg/mL to about 250 mg/mL, from about 70 mg/mL to about 250 mg/mL, from about 80 mg/mL to about 250 mg/mL, from about 90 mg/mL to about 250 mg/mL, from about 100 mg/mL to about 250 mg/mL, from about 150 mg/mL to about 250 mg/mL, or from about 200 mg/mL to about 250 mg/mL.

Where a polymerization method is utilized to form the spectrally encoded polymeric microbeads, a suitable polymerization initiator (e.g., a photoinitiator or thermal initiator) may be utilized which is compatible with the polymerizable components and the polymerization conditions. For example, where a UV polymerization process is utilized, a suitable initiator may include a compound that, when exposed to UV light, undergoes a photoreaction, producing reactive species that are capable of initiating polymerization. Exemplary photoinitiators may include, e.g., acetophenones, benzyl and benzoin compounds, benzophenone, cationic photoinitiators, and thioxanthones. In some embodiments, a photoinitiator such as 2-hydroxy-4′-(2-hydroxyethoxy)-2-methylpropiophenone (Irgacure® 2959) or lithium phenyl-2,4,6-trimethylbenzoylphosphinate (LAP) is utilized. Suitable thermal initiators may include, for example, azo compounds, peroxides or hydroperoxides, persulfates, and the like.

The step of forming droplets from the solution may include, for example, contacting a hydrophilic solution containing the polymerizable component with a hydrophobic solvent (e.g., mineral oil or water-immiscible organic solvent, e.g., octanol) such that droplets are formed. Alternatively, a hydrophobic solvent (e.g., mineral oil or water-immiscible organic solvent, e.g., octanol) can be used to form the solution containing the polymerizable component, and droplets can be formed by contacting the solution with a hydrophilic carrier fluid (e.g., water). These steps may be accomplished, for example, by introducing the solution containing the polymerizable component into a flowing stream of the carrier fluid. Any suitable device and/or method for droplet formation may be utilized to form droplets in the context of the present disclosure, including, e.g., the utilization of flow focusing nozzles. See, e.g., Ward et al. (Electrophoresis, 2005, 26:3716-3724), the disclosure of which is incorporated by reference herein. The droplet size may be modulated by adjusting the pressure used to form the droplet, e.g., at the interface of the solution and the hydrophobic carrier fluid. In addition, droplet size may be modulated by adjusting the geometry, e.g., size and shape, of the microfluidic device channels. One or more stabilizers or surfactants may be added to one or more of the fluids to prevent droplet merging and sticking of droplets to the walls of the microfluidic device. Suitable surfactants may include, for example, Abil® EM90 (a silicon based emulsifier; CAS No. 144243-53-8) and Span™ 80 (CAS No. 1338-43-8), among others.

In some embodiments, monomer input solutions for microbead synthesis contain purified water, monomer (e.g., 700 MW PEG-diacrylate, 40-60% w/w), initiator (e.g., photoinitiator Irgacure® 2959, 1-10% w/w), Sigma-Aldrich), and lanthanide nanoparticles at various concentrations. Droplets can be formed by introducing the input solutions in a continuous flowing stream of hydrophobic fluid (e.g., light mineral oil), with or without surfactants to reduce or eliminate merging of droplets (e.g., Abil® EM90 (Evonik Industries, Germany), Span™ 80 (Sigma-Aldrich), 0.05%-5% w/w).

The step of subjecting the droplets to polymerization conditions may include, for example, exposing the droplets to UV radiation or elevated temperatures to initiate polymerization. Other known polymerization methodology can be employed, provided that it is compatible with the polymers and/or monomer components to be polymerized. Examples of such methodology include, but are not limited to, thiol-ene polymerization, redox-initiated polymerization, and controlled radical polymerization processes such as reversible addition-fragmentation chain transfer (RAFT) polymerization, atom transfer radical polymerization (ATRP), and nitroxide-mediated polymerization (NW). See also, e.g., Piskin et al. (J. of Biomaterials Science Polymer Edition 1994, 5: 451-471), the disclosure of which is incorporated by reference herein.

In some embodiments, droplets are exposed to radiation (e.g., UV radiation) by localizing the radiation exposure onto a microfluidic device such that the droplets are only irradiated after they have been formed on the microfluidic device and before they exit the microfluidic device. Radiation localization may be achieved using an inverted microscope by mounting the microfluidic device on the microscope stage. For example, UV illumination may occur through the objective onto a very small area and an additional aperture within the microscope UV light path may further restrict the UV irradiation to a specific area of the microfluidic device. Alternatively, UV illumination may occur after the droplets exit the microfluidic device.

Methods which do not require polymerization may also be used to form the spectrally encoded polymeric microbeads. For example, a polymer precipitation method may be utilized in which a pre-formed polymer (e.g., a mid- to high-molecular weight polymer) is dissolved in a suitable solvent (e.g., water) along with dispersed lanthanide nanoparticles. Droplets of this solution can be formed by introducing the solution into an immiscible carrier fluid (e.g., a hydrophobic carrier fluid, e.g., mineral oil). The immiscible carrier fluid and polymer should be selected such that the polymer does not dissolve in the immiscible carrier fluid, and the immiscible carrier fluid is capable of accepting the solvent leaching from the droplet as the polymeric microbead is formed through precipitation. Additional solvent-immiscible carrier fluid combinations may include, e.g., dichloromethane as a solvent and poly(vinyl alcohol) (PVA) as an immiscible carrier fluid. Microbead preparation methods utilizing a dichloromethane-poly(vinyl alcohol) (PVA) combination are described, for example, in Berkland et al. (Journal of Controlled Release, 2002, 73:59-74; Journal of Controlled Release, 2004, 94:129-141), the disclosures of which are incorporated by reference herein.

The steps of mixing at least two fluids can occur either before or after droplet formation depending on the particular microfluidic device architecture utilized. For example, where a herringbone type mixing architecture is utilized the two fluids may be mixed prior to droplet formation. Alternatively, where a zig-zag type mixing architecture is utilized droplets containing unmixed lanthanide nanoparticles may be formed and subsequently mixed to distribute the lanthanide nanoparticles within a droplet. Accurate programming of spectral codes for the spectrally encoded microbeads may be facilitated by precisely controlling the flow from each of the lanthanide nanoparticle fluid inputs as previously described, for example, in U.S. Pat. No. 10,241,045.

### Functionalization of Microbeads for Attachment of Oligonucleotides

In some embodiments, microbeads are functionalized with reactive groups for conjugation of capture polynucleotides. Typically, a population of microbeads having the same spectral signature (e.g., a population ranging up to thousands of beads) is carried through one or more functionalization steps such that (i) each microbead in the population having the same spectral signature is conjugated to a spectral code identification oligonucleotide of the same sequence, and (ii) the number of spectral code identification oligonucleotide conjugated to each microbead in the population is substantially the same. This process can be conducted, for example, with a microfluidic device having an in-line fraction collector for pooling of microbead populations by spectral signature. Sub-populations may be reserved for use in different chemical steps, or in some embodiments, for use in distributing to an arrayed configuration such that the majority of compartments contain a single microbead having spectral signature that differs from the spectral signatures of microbeads in the other compartments.

In some embodiments, microbeads are functionalized with reactive groups that can then be conjugated to capture polynucleotides via one or more click reactions. As used herein, “click reaction” refers to a chemical reaction characterized by a large thermodynamic driving force that usually results in irreversible covalent bond formation. Examples of click reactions include thiol-ene reactions, such as the Michael addition of a thiol to a maleimide or other unsaturated acceptor; [3+2] cycloadditions, such as the Huisgen 1,3-dipolar cycloaddition reaction of an azide and an alkyne; [4+1] cycloaddition reactions between an isonitrile and a tetrazine; the Staudinger ligation between an azide and an ester-functionalized phosphine or an alkanethiol-functionalized phosphine; Diels-Alder reactions (e.g., between a furan and a maleimide); and inverse electron demand Diels-Alder reactions (e.g., between a tetrazine and a dienophile such as a strained transcyclooctene).

In some embodiments, the microbeads contain carboxylate groups for bonding to amine-functionalized capture polynucleotides. For example, acrylate groups in microbeads can be coupled to a thiol-functionalized carboxylic acid (e.g., 3-mercaptopropionic acid) in the presence of a base such as pyridine, N,N-diisopropylamine (DIPEA), or 1,8-diazabicyclo[5.4.0]-undec-7-ene (DBU). The carboxylates can then be reacted with amine-functionalized oligonucleotides using one or more coupling reagents such as a carbodiimide (e.g., N,N′-dicyclohexylcarbodiimide (DCC), 1-ethyl-3-(3-dimethylaminopropyl)-carbodiimide (EDC), a phosphonium salt (HOBt, PyBOP, HOAt, etc.), an aminium/uronium salt, a pyridinium coupling reagent (e.g., Mukaiyama's reagent, pyridinium tetrafluoroborate coupling reagents, etc.), a polymer-supported reagent (e.g., polymer-bound carbodiimide, polymer-bound TBTU, polymer-bound 2,4,6-trichloro-1,3,5-triazine, polymer-bound HOBt, polymer-bound HOSu, polymer-bound IIDQ, polymer-bound EEDQ, etc.), or the like (see, e.g., El-Faham, et al. Chem. Rev., 2011, 111(11): 6557-6602; Han, et al. Tetrahedron, 2004, 60:2447-2467).

In some embodiments, the capture oligonucleotide includes a clickable moiety for reaction with a complementary clickable moiety on a microbead. As used herein, a “clickable moiety” refers to a functional group that is capable of forming a covalent bond via a click reaction, such as an azide, an alkyne, a phosphine, a thiol, a maleimide, an isonitrile, or a tetrazine. In some embodiments, each clickable moiety is independently selected from the group consisting of an azide, an alkyne, and a phosphine. In some embodiments, the microbeads contain an alkyne moiety for bonding to azide-functionalized capture polynucleotides. For example, the amine groups in microbeads formed using PEG monoacrylamide-monoamine (PEG-AM) can be coupled to a carboxylate-functionalized alkyne (e.g., 4-pentynoic acid) using a carbodiimide reagent such as 1-ethyl-3-(3-dimethylaminopropyl)carbodiimide hydrochloride (EDC). The alkynes can then be reacted with azide-functionalized oligonucleotides using a suitable catalyst (e.g., a copper salt such as copper (II) acetate, copper (II) sulfate, copper (1) bromide, or copper (1) iodide) and optional reducing agents (e.g., sodium ascorbate) and ligands (e.g., tris[(1-benzyl-1H-1,2,3-triazol-4-yl)methyl]amine (TBTA), tris(2-benzimidazolylmethyl)amine, tris(3-hydroxypropyltriazolylmethyl)amine, or the like.

In some embodiments, a crosslinking reagent is used for attachment of the capture polynucleotides to the microbeads. The crosslinking reagents can react to form covalent bonds with functional groups in the capture oligonucleotides and on the microbead surfaces (e.g., a primary amine, a thiol, a carboxylate, a hydroxyl group, or the like). Crosslinkers useful for attaching capture oligonucleotides to microbeads include homobifunctional crosslinkers, which react with the same functional group in the oligonucleotide and the bead, as well as heterobifunctional crosslinkers, which react with functional groups in the bead and the oligonucleotide wherein the functional groups differ from each other.

Examples of homobifunctional crosslinkers include, but are not limited to, amine-reactive homobifunctional crosslinkers (e.g., dimethyl adipimidate, dimethyl suberimidate, dimethyl pimilimidate, disuccinimidyl glutarate, disuccinimidyl suberate, bis(sulfosuccinimidyl) suberate, bis(diazo-benzidine), ethylene glycobis(succinimidylsuccinate), disuccinimidyl tartrate, disulfosuccinimidyl tartrate, glutaraldehyde, dithiobis(succinimidyl pro-pionate), dithiobis-(sulfosuccinimidyl propionate), dimethyl 3,3′-dithiobispropionimidate, bis 2-(succinimidyl-oxycarbonyloxy)ethyl-sulfone, and the like) as well as thiol-reactive homobifunctional crosslinkers (e.g., bismaleidohexane, 1,4-bis-[3-(2-pyridyldithio)propionamido]butane, and the like). Examples of heterobifunctional crosslinkers include, but are not limited to, amine- and thiol-reactive crosslinkers (e.g., succinimidyl 4-(N-maleimidomethyl)-cyclohexane-1-carboxylate, m-maleimidobenzoyl-N-hydroxysuccinimide ester, succinimidyl-4-(p-maleimidophenyl)butyrate, N-(y-maleimidobutyryloxy)succinimide ester, N-succinimidyl(4-iodoacetyl) aminobenzoate, 4-succinimidyl oxycarbonyl-a-(2-pyridyldithio)-toluene, sulfosuccinimidyl-6-a-methyl-a-(2-pyridyldithio)-toluamido-hexanoate, N-succinimidyl-3-(2-pyridyldithio) propionate, N-hydroxysuccinimidyl 2,3-dibromopropionate, and the like).

Reaction mixtures for attaching capture oligonucleotides can contain additional reagents of the sort typically used in bioconjugation reactions. For example, in certain embodiments, the reaction mixtures can contain buffers (e.g., 2-(N-morpholino)ethanesulfonic acid (MES), 2-[4-(2-hydroxyethyl)piperazin-1-yl]ethanesulfonic acid (HEPES), 3-morpholinopropane-1-sulfonic acid (MOPS), 2-amino-2-hydroxymethyl-propane-1,3-diol (TRIS), potassium phosphate, sodium phosphate, phosphate-buffered saline, sodium citrate, sodium acetate, and sodium borate), cosolvents (e.g., dimethylsulfoxide, dimethylformamide, ethanol, methanol, tetrahydrofuran, acetone, and acetic acid), salts (e.g., NaCl, KCl, CaCl₂), and salts of Mn²⁺ and Mg²⁺), detergents/surfactants (e.g., a non-ionic surfactant such as N,N-bis[3-(D-gluconamido)propyl]cholamide, polyoxyethylene (20) cetyl ether, dimethyldecylphosphine oxide, branched octylphenoxy poly(ethyleneoxy)ethanol, a polyoxyethylene-polyoxypropylene block copolymer, t-octylphenoxypolyethoxyethanol, polyoxyethylene (20) sorbitan monooleate, and the like; an anionic surfactant such as sodium cholate, N-lauroylsarcosine, sodium dodecyl sulfate, and the like; a cationic surfactant such as hexdecyltrimethyl ammonium bromide, trimethyl(tetradecyl) ammonium bromide, and the like; or a zwitterionic surfactant such as an amidosulfobetaine, 3-[(3-cholamidopropyl)dimethyl-ammonio]-1-propanesulfonate, and the like), chelators (e.g., ethylene glycol-bis(2-aminoethylether)-N,N,N′,N′-tetraacetic acid (EGTA), 2-({2-[bis(carboxymethyl)amino]ethyl} (carboxymethyl)amino)acetic acid (EDTA), and 1,2-bis(o-aminophenoxy)ethane-N,N,N′,N′-tetraacetic acid (BAPTA)), and reducing agents (e.g., dithiothreitol (DTT), β-mercaptoethanol (BME), and tris(2-carboxyethyl)phosphine (TCEP)). Buffers, cosolvents, salts, detergents/surfactants, chelators, and reducing agents can be used at any suitable concentration, which can be readily determined by one of skill in the art. In general, buffers, cosolvents, salts, detergents/surfactants, chelators, and reducing agents are included in reaction mixtures at concentrations ranging from about 1 μM to about 1 M. For example, a buffer, a cosolvent, a salt, a detergent/surfactant, a chelator, or a reducing agent can be included in a reaction mixture at a concentration of about 1 μM, or about 10 μM, or about 100 PM, or about 1 mM, or about 10 mM, or about 25 mM, or about 50 mM, or about 100 mM, or about 250 mM, or about 500 mM, or about 1 M.

The reactions are conducted under conditions sufficient to install the capture oligonucleotides on the microbeads. The reactions can be conducted at any suitable temperature. In general, the reactions are conducted at a temperature of from about 4° C. to about 40° C. The reactions can be conducted, for example, at about 25° C. or about 37° C. The reactions can be conducted at any suitable pH. In general, the reactions are conducted at a pH of from about 4.5 to about 10. The reactions can be conducted, for example, at a pH of from about 5 to about 9. The reactions can be conducted for any suitable length of time. In general, the reaction mixtures are incubated under suitable conditions for anywhere between about 1 minute and several hours. The reactions can be conducted, for example, for about 1 minute, or about 5 minutes, or about 10 minutes, or about 30 minutes, or about 1 hour, or about 2 hours, or about 4 hours, or about 8 hours, or about 12 hours, or about 24 hours, or about 48 hours, or about 72 hours. Other reaction conditions may be employed in the methods of the invention, depending on the composition of the microbeads and the reagents used for installing the capture oligonucleotides.

### Determining Lanthanide Spectral Signatures

The combined luminescent signals generated from lanthanide nanoparticles provide a unique pattern, i.e., signature, that is specific to a microbead. In some embodiments, each of the lanthanide spectral signatures comprises a europium (Eu) signal, a dysprosium (Dy) signal, a samarium (Sm) signal, a cerium (Ce) signal, a terbium (Tb) signal, a lanthanum (La) signal, a praseodymium (Pr) signal, a neodymium (Nd) signal, a gadolinium (Gd) signal, a holmium (Ho) signal, an erbium (Er) signal, a thulium (Tm) signal, an ytterbium (Yb) signal, or a combination thereof. In some embodiments, each of the microbeads comprises a plurality of lanthanide nanoparticles. In some embodiments, the lanthanide nanoparticles comprise a lanthanide-doped host lattice. In some embodiments, the host lattice is yttrium orthovanadate, lanthanum phosphate, or a combination thereof. In some embodiments, each of the microbeads further comprises a polymer coating covering the lanthanide nanoparticles, and wherein the capture oligonucleotides are covalently bonded to the polymer coating. In some embodiments, a plurality of the microbeads contains at least 100 lanthanide spectral signatures. In some embodiments, the plurality of microbeads contains at least 1000 lanthanide spectral signatures.

In general, each of the spectral signatures employed in an assay contains signals generated from predetermined amounts of two or more lanthanides (e.g., two or more Eu-, Dy-, Sm-, Ce-, Tb-, La-, Pr-, Nd-, Gd-, Ho-, Er-, Tm-, Yb-containing materials such as nanoparticles). Lanthanide materials in the microbeads can be excited with UV light (e.g., 275 nm or 292 nm) and emitted luminescent signals can be detected in the range of 400-800 nm (e.g., 435 nm, 474 nm, 527 nm, 536 nm, 546 nm, 572 nm, 620 nm, 630 nm, 650 nm, or 780 nm). A signal in a spectral signature may be measured as an absolute value or as a ratio of the signal to another reference signal. As a non-limiting example, a set of unique spectral signatures can be prepared with microparticles that contain europium-dope yttrium orthovanadate (YVO₄:Eu) to generate a reference signal and varying amounts of YVO₄:Dy, YVO₄:Sm, YVO₄:Tm, and LaPO₄:CeTb.

In an array configuration as described herein, a lanthanide spectral signature of a microbead is typically determined via deep UV imaging as described above of the compartments of the arrayed configuration. The spectral signature of the microbeads present in the single-cell analysis compartments and the spatial position of those compartments in the array can thus be determined. In some embodiments, lanthanides embedded in different host matrices excite at low energy light, e.g., 980 nm IR, and emit in visible light. In applications employing such “upconverting” lanthanide:host matric combinations, the spectral signature is determined via infrared imaging.

### Capture Oligonucleotides

Microbeads comprising a lanthanide spectral signature as used in the methods and devices described herein are joined to a population of capture oligonucleotides. In some embodiments, this is achieved via direct synthesis of capture oligonucleotides that contain both the spectral signature domain and the capture domain, and any other desired sequences. In other embodiments, an extension reaction, e.g., employing Klenow, can be performed to obtain the desired full-length capture oligonucleotides. The population of capture oligonucleotides comprises capture oligonucleotides that have at least two domains. One of the domains comprise a spectral signature identifier sequence. The spectral signature identifier sequence of the population is specific to a single lanthanide spectral signature, and hence, specific to a single microbead that is evaluated in the assay, and is incorporated into the individual capture oligonucleotides present in the population immobilized to the bead. The spectral signature identifier sequence is sufficiently uniform throughout the population to allow the identifier sequence to be unambiguously paired with the spectral signature and thus, unambiguously paired with a single bead that is evaluated in the assay. The edit distance, the number of single base edits required to make one sequence into another, of the spectral signature identifier sequences that distinguish the different spectral signatures typically have an edit distance of at least 3, 4, 5, or 6, greater, when compared to one another.

As appreciated by one on the art, the length of the sequence can be designed taking into account the number of spectral codes there are for the individual microbeads employed in an analysis. In some embodiments, the spectral signature identifier sequence is from 6 to 12 nucleotides in length, i.e., from 6, 7, 8, 9, 10, 11, or 12 nucleotides in length. In some embodiments, the spectral signature identifier sequence is 6, 7, 8, 9, or 10 nucleotides in length. In some embodiments, the spectral signature identifier sequence may be greater than 12, nucleotide in length, e.g., 15 to 25 nucleotides in length, or longer, e.g., up to 50 nucleotides in length; or greater than 50 nucleotides in length. In some embodiments, a spectral signature identifier sequence may be disrupted rather than contiguous.

A second domain contained in the capture oligonucleotide is a capture domain that targets nucleic acid populations of interest in a single cell. The capture domain may vary among members of the population of capture oligonucleotides attached to a microbead to target different nucleic acids in the single cell. In other embodiments, the capture domain is essentially the same, i.e., individual capture domains in the population target the same nucleic acids of interest. Thus, for example, in some embodiments, the capture oligonucleotides may contain capture domains that target particular RNAs of interest, e.g., encoding one or more cytokines or particular classes of polypeptides; or particular DNA molecules of interest, e.g., a target genes for genotype or haplotype analysis. In some embodiments, the capture oligonucleotides may contain a capture domain that is shared by the oligonucleotides in the population, e.g., a poly(T) or poly(U) tract to bind poly(A)⁺ RNA. Thus, the capture domain is complementary, or substantially complementary, to a target nucleic acid population of interest and selectively hybridizes to the target nucleic acid population such that the target population is bound to the microbead.

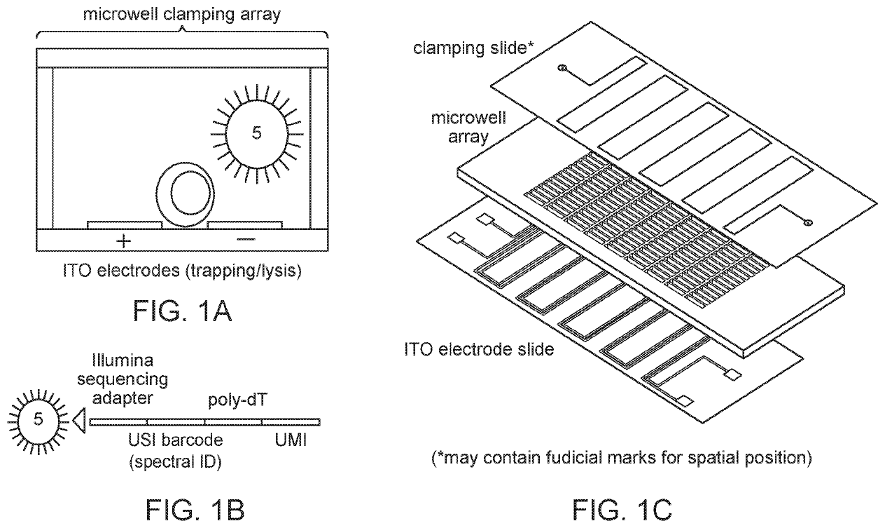

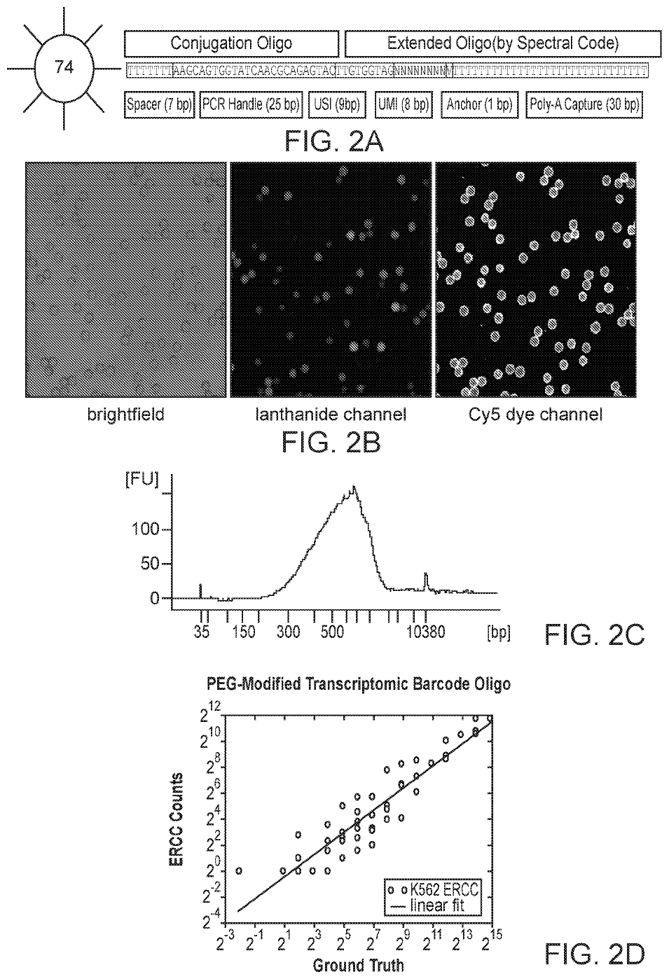

Capture oligonucleotides may comprise sequences in addition to the spectral signature identifier domain and capture domains, for example, a primer binding site (also referred to as a PCR handle in FIGS. 1 and 2) for amplification, a unique molecular identifier sequence for quantification of specific transcripts, and/or a spacer sequence at the end of the oligonucleotide that is attached to the microbead. In some embodiments, capture oligonucleotide may comprise sequences employed for preparing libraries for high throughput sequence, (e.g., primer sequences for high throughput sequencing library preparation).

Single cells in the compartments of the array configuration are permeabilized or lysed in the compartment and the nucleic acids are hybridized with the capture oligonucleotides immobilized on the beads. After a time sufficient for hybridization, the beads are then processed for sequence analysis of the target nucleic acid population, e.g., by amplifying the nucleic acids hybridized to the capture oligonucleotides. In some embodiments, the microbeads in single-cell analysis compartments are collected, which may occur prior to, or after, processing of the bead-bound target nucleic acids (hybridized to the capture oligonucleotides), for sequencing. The target nucleic acids from the single cell analysis compartments are pooled for high throughput sequencing.

Various single cell assays can be used in for evaluating a sequence-based single cell parameter. Exemplary assays include RNA sequencing, including, but not limited to, sequencing of mRNA, and other RNA populations of interest such as miRNA, snRNA, lncRNA and the like; and genomic DNA sequencing, including, but not limited to, haplotyping and phase determination, genotyping, intron and/or exon sequences. In some embodiments, target genomic nucleic acids can be analyzed by ATAC-Seq, e.g., by capturing mosaic Tn5 sequences. In some embodiments, nucleic acid targets may be a single target, e.g., analysis targeting a specific gene, or may involve multiple targets, e.g., analysis of a plurality of genes of interest. In still other embodiments, target nucleic acids that are analyzed are from transgenic organisms or CRISPR-modified organisms. In other embodiments, target nucleic acids, e.g., T cell receptor alpha and beta chains and/or Major Histocompatability Complex targets can be evaluated for immunoclassification purposes. In some embodiments, antibody nucleic acids may be targeted to evaluate an antibody repertoire. In still other embodiments, mitochondrial DNA may be targeted by capture oligonucleotides for sequence analysis, or nucleic acids contained in exosomes may be targeted for sequence analysis. Various assays describing DNA genotyping, genomic sequences and large genome characterization via exon sequences, are detailed, e.g., in Gnirke, Andreas, et al., Nat. Biotechnol. 27: 182-189, 2009; Hodges et al., Nat. Genetics: 39:1522, 2007; Okou, et al., Nat. Methods 4:907-909, 2007; Porreca, et al., Nat. Methods 4:931, 2008; and Turner et al., Nat. Methods 6:315, 2009.

In some embodiments, the capture oligonucleotides target RNA to evaluate expression, e.g., the transcriptosome of a single cell, or RNAs of interest. RNA expression using single cell RNA sequencing (RNA-Seq) is described, e.g., by Tang et al., Nat. Methods 6:377-382, 2009; Ramskolod et al., Nat. Biotechnology 30:777-782, 2012; Macosko et al., Cell 161: 1202-1214, 2015; WO2016/040476; Klein et al., Cell 161:1187-1201, 2015; WO2016168584; Zheng, et al., Nature Biotechnology 34:303-311, 2016; Zheng, et al., Nat. Commun. 8: Article number 14049, 2017; WO 2014210353; Zilionis, et al., Nat Protoc. 12:44-73, 2017; Cao et al., Science 357:661-667, 2017; and Rosenberg et al., 2017, “Scaling single cell transcriptomics through split pool barcoding” bioRxiv preprint Feb. 2, 2017. Both unbiased and targeted approaches may be employed.

Sequence analysis of the target nucleic acids obtained via hybridization to the capture domain provides the sequence of the capture oligonucleotide that includes both the spectral signature identifier sequence of the plurality of capture oligonucleotides as well as the sequences of individual nucleic acid molecules bound to the capture domain. As explained above, the identifier sequence provides the ability to associate the sequence data with a spectral signature in a specific 1:1 linkage and hence, the cell from which the sequence data is derived and phenotypic parameters measure for that cell. Thus, for example, in some embodiments, an RT reaction is performed with the oligonucleotides attached to the beads to provide covalent stability on the bead. An amplification reaction, e.g., PCR can then be performed top provide amplicons that can be pooled for sequence analysis.

Phenotypic assays measure a parameter that does not require direct sequencing of nucleic acids. Thus, for example, phenotypic assays include any analysis that images or otherwise detects a signal from a cell in the single-cell analysis compartments. Phenotypes that can be assayed include, but are not limited to, imaging individual cells across a statistically significant population for morphological characteristics, functional behaviors (e.g., migration, movement, growth), anatomical changes (e.g., spindle generation, cytoskeletal reorganization, axon elongation), signaling behaviors (e.g., surface marker presence, paracrine synapse, paracrine cytokine or signaling molecule efflux), and/or dynamic response (e.g., ‘tracking’ intracellular protein compartmentalization over time). Additional phenotypic assays that can be assess include non-imaging assays such as cellular density assessment in magnetic levitation, deformation analysis to applied force, or electrical characterization (e.g., patch clamp and ion flux assays). In some embodiments, polypeptide profiles of a single cell may be evaluated using a panel of barcoded antibodies. For example, cytokine profiles may be determined by incubating the single cells present in the single-cell analysis compartments with a panel of bar-coded antibodies to cytokines of interest. Cytokine expression profiles can be evaluated by sequence analysis to identify the antibodies in the panel that bind to cytokines produced by a single cell.

In some embodiments, the expression of a protein or panel of proteins is assessed in individual cells using antibody-based detection methods; or other polypeptides or binding agents, that specifically bind to a molecule of interest in a cell. Illustrative assays include, but are not limited to, proximity ligation assay and proximity extension assays; immunofluorescence assays and other antibody-based assays. Any protein or combination of proteins can be detected including intracellular proteins, secreted proteins, and proteins expressed on the cell surface. In some embodiments, lipids and carbohydrates are assessed. Thus, for example, in particular embodiments, glycosaminoglycan, proteoglycan, phospholipid, or any other cellular membrane component can be assessed.

In some embodiments, a phenotypic analysis comprises detecting one or more signals from detection reagents added to the single cell analysis compartment. Examples of such reagents include intracellular dyes or stain, or detection reagents labeled with a detectable label, e.g., fluorescent label, a particle label, an oligonucleotide label, a small molecule, or other detection reagent. In some embodiments, reagents such as a peptide that is used to evaluate cellular reactivity to that peptide may be immobilized on the microbeads. In some embodiments, a reagent may be immobilized on the cellular compartments. In some embodiments, a genetically engineered fluorescent tag, e.g., GFP, can be linked to proteins for detection, e.g., tracking one or more proteins over time.

In some embodiments, a phenotypic assay measures a parameter such as cell motility, cell morphology, and/or perturbation of a cell by an agent of interest. Such assay can comprise use of a signal-generating reagent, e.g., a detection reagent labeled with a detectable label, such as a fluorescent dye, chemiluminescent agent, radioisotope, or other label. In some embodiments, the phenotypic parameter, e.g., cell morphology, is assessed by image analysis without the use of a detectable reagent. In some embodiments, cellular perturbations induced by small molecule drugs may be evaluated, e.g., phenotypic changes, including changes in morphology and the like, may be assessed.

Illustrative phenotypic assays include, but are not limited to, calcium flux in response to a stimulus, monitoring of cytokine secretion, e.g., from CD4⁺ stimulated T-cells (stimulated non-specifically with drug cocktails e.g., PMA lonomycin, or specifically in co-culture). For example, cytokines (e.g., INF-gamma, IL2, IL4, IL6, etc.) can be profiled per cell to generate a functional phenotype per cell of cytokines secreted per time point, e.g., using a coupled antibody barcode approach similar to Fan, et al., Nature Biotechnol. 26: 1373, 2008 and Lu et al, Proc. Natl. Acad. Scie USA 112:E607-E615, 2015. Additional phenotypic assays may also include dynamic monitoring of protein translocation and movement within a cell using a fluorescent stain or transgenic fluorescent protein coupled to the protein-of-interest. For example, nuclear factors (e.g., NF-kB), transcription factors (e.g., GATA3) or cytoplasmic assemblies can be monitored by fluorescent intensity localized within the cell over time via time-course imaging in this assay similar to Lane et al., Cell Systems 4:458469, 2017.

In some embodiments, the phenotypic assay is performed at the same time as an imaging assay that detects the spectral signature of the microbeads in a array configuration.

A phenotypic assay can be performed multiple times, e.g., to measure a time course of cellular responses to a stimulus.

Any number of devices can be used for the array configuration to assay phenotypic and spectrally-encoded microbeads for association with sequencing information. FIG. 1 depicts an illustrative device. FIG. 1, panel A shows a spectrally encoded microbead (spectral code ‘5’) and a single cell in a microfluidic microwell compartment. The spectral microbead serves as the link between an imaging-based phenotype, for example, dynamic cellular imaging; and a high throughput sequencing analysis of an aspect of cellular function (in this example, the microbead serves as a solid phase support for bead-based RNA sequencing). FIG. 1, panel B provides an illustration of the spectrally encoded microbead and its spectral signature identifier sequence (referred to in FIG. 1 as a unique spectral identifier (USI) The spectral code of the bead is specific to a given spectral signature identifier sequence (“spectral ID”). FIG. 1 provides a stack view of an illustrative integrated device; the clamp is not shown. The DEP ITO slide for electrical lysis is employed in this particular example, and is not necessarily present in all embodiments of the invention.

Devices employing single cell compartments other than microwells may be employed, e.g., droplets, including arrayed or patterned droplets; superhydrophobic/hydrophilic spot arrays, micropores or nanopores, trap and pillar arrays, vertical chamber arrays and the like can serve as compartments for performing single-cell multi-parameter analysis as described herein.

### Single Cells

Single cells from any source, including any plant, animal, or microorganism may be analyzed in accordance with the methods of the invention. In some embodiments, cells are eukaryotic cells, including, but not limited to, yeast and fungi cells, plant cells, avian cells, mammalian cells, and the like. In some embodiments, the cells are mammalian cells, e.g., human cells. In some embodiments, the cells are cancer cells, stem cells, neurological cells, peripheral blood mononuclear cells, lymphocytes, or cells from a cell line. In some embodiments, the cells are obtained from a tissue e.g., a human tissue. In some embodiments, the cells are obtained from a tumor, e.g., a human tumor. In some embodiments, single cells from transgenically modified organisms may be evaluated, e.g., for CRIPSPR-based screening.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims.

### Example 1. Tandem RNA-Seq and Phenotypic Analysis

FIG. 2 illustrates microbead/capture oligonucleotide organization and analyzing gene expression profiles by RNA-Seq in tandem with imaging. Panel A shows a schematic of sequence construction for transcriptomic (poly(A)+RNA) capture on a microbead. Each spectrally encoded bead code (shown as ‘74’ as an example spectral code) contains a region designated in the schematic a the “Conjugation Oligo” sequence, which contains a PCR primer binding site (referred to as a “PCR handle” in the schematic) and attachment regions for conjugation to the spectral microbead. The region of the oligonucleotide referred to as the “Extended Oligo” oligonucleotide sequence in the schematic contains a spectral signature identifier sequence (referred to as a “Unique Spectral Index” or “USI sequence” in the schematic) specific for each individual spectral code to link massively parallel sequencing data to the spectral code. This region may optionally contain a unique molecular index for molecular counting and transcriptomic or other capture regions for recruiting cellular transcriptomics (poly-T capture site as shown in Panel A). Panel B shows images of spectrally encoded beads in brightfield, an example of a visible emission channel to devolve lanthanide spectral codes, and a fluorescent emission channel (Cy5). The Cy5 conjugation signal on each bead showed specific hybridization of a complementary oligonucleotide to oligonucleotide conjugated on the spectral beads. These images confirmed the ability to conjugate oligonucleotides of interest on the spectrally encoded beads and subsequently perform hybridization (in this case with a complementary oligonucleotide containing a Cy5 probe), which is necessary for recruiting transcripts and other desired cellular nucleotides specifically to the bead surface. Panel C provides an example of bioanalyzer (Agilent High Sensitivity Bioanalyzer)-size fragment traces of massively parallel sequencing libraries from RNA recruitment and sequencing preparation from spectrally encoded beads. Cellular mRNA was recruited to the bead surface using Poly(A) capture tracts as shown in Panel A, hybridized, pooled and washed. Subsequently, reverse transcription (RT), template switching PCR, and tagmentation were performed to convert recruited RNA to cDNA, amplify products, and size-restrict fragments for NGS and RNA-Seq analysis. This example bioanalyzer trace is post-tagmentation with sequencer-ready fragments of sizes of about 150-750 bp prepared from K562 (a human leukemia cell line) mRNA recruited to the spectrally encoded micro beads. Panel D illustrates sequencing enrichment using spectrally encoded beads for K562 RNA+ERCC RNA spike-in controls for ˜1000 spectral encoded beads. The x-axis corresponds to ERCC molecular enrichment counts for 92 ERCC synthetic RNA species (black dots on plot) in the known transcriptomic mixture hybridized to spectrally encoded beads. The y-axis corresponds to resultant ERCC molecular counts for the 92 ERCC synthetic RNA species as a spike-in to the cellular RNA recruitment. Black dots correspond to each ERCC species (each species of different concentration, length and GC bias) and the fit line represents how well sequencing data matches expected distribution. R2 value=0.8558 highlighting good agreement at low sequencing depth (MiSeq). Spectral beads were conjugated with a PEG linker and oligo sequences as shown in panel A for transcriptomic recruitment, capture and processing.

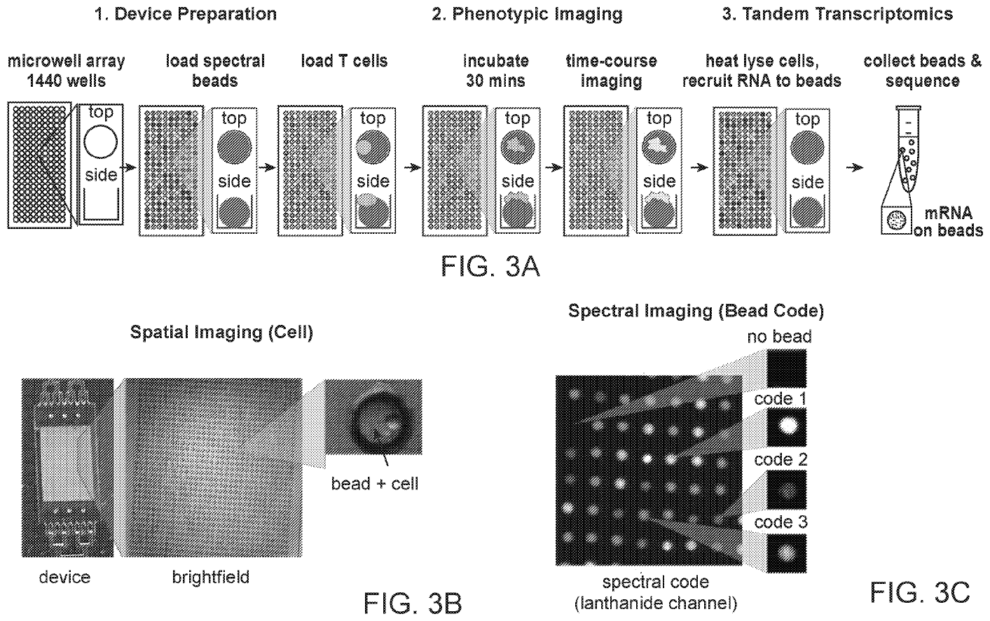

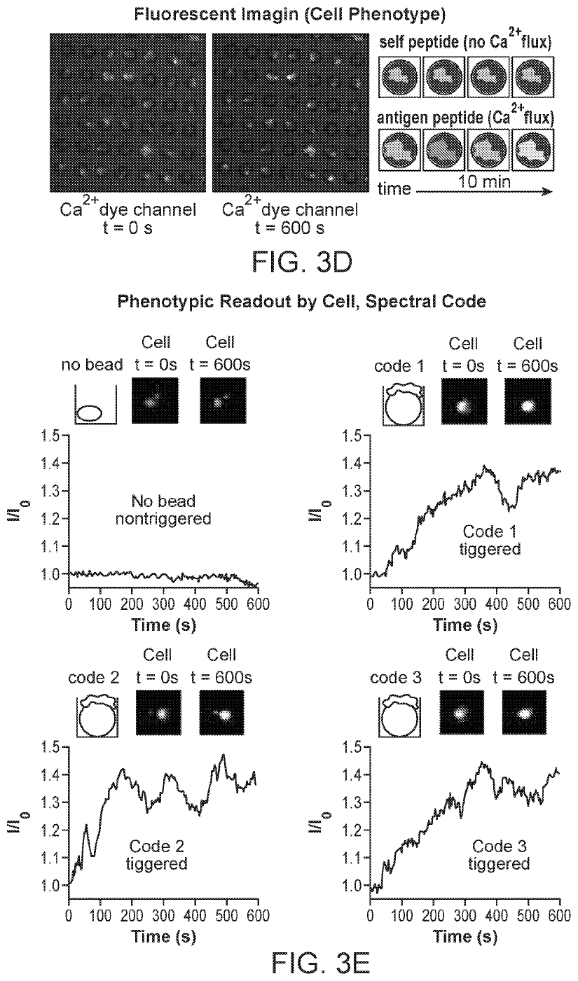

An example of an image-based phenotypic assay is shown in FIG. 3, Panels A-E. Panel A provides an overview schematic of a specific embodiment of tandem cellular phenotyping and transcriptomic analysis (RNA-Seq) on the same cell using spectrally encoded microbeads. The workflow as shown uses hybridoma (or primary) T cells in which a 1440 passive microwell array, a microfluidic device, are used for bead and cell co-encapsulation. Beads are loaded first into the array, allowed to settle, followed by cell loading and co-incubation. In this particular assay, T cells are activated or inactivated (resulting in an associated observable Ca²⁺ flux response) by either self-peptide or antigen peptide. These peptides can either be bound to the bead surface in addition to oligo capture sequences for transcriptomics (as illustrated here) or associated with a spatially encoded array. This is a demonstration of a perturbation-based cellular phenotype. After associated variable activation, the T cells with their associated beads are imaged under time-course for observation of a dynamic phenotype (Ca²⁺ flux) associated with a fluorescent dye. During this imaging process, images in brightfield and associated lanthanide spectral channels are taken to ascertain bead, cell position and bead spectral code. After phenotyping cells are lysed via heat, RNA is recruited to the beads, and beads are collected for sequencing. Panel B shows imaging in the brightfield channel of the passive microwell device. The inset shows an example of a well with co-encapsulated spectral microbead and cultured T cell. Panel C provides an example of spectral code imaging (example visible channel emission after deep UV excitation) used to decode microbead spectral codes as indicated (during the experiment, microbead imaging was conducted over nine spectral encoding visible emission channels. Panel D shows a time-course phenotypic imaging using fluorescent microscopy. A dye associated with Ca²⁺ flux was imaged using fluorescence emission per each well in the microwell array (and spatially linked to cellular presence in brightfield and microbead code in the lanthanide emission channels). Ca²⁺ signal flux was tracked per cell over a 10-minute period with multiple intervals (shown in Panel E). In this example, certain cells were perturbed by self-peptide, which results in no Ca²⁺ flux, and certain cells were perturbed by antigenic peptide, which results in an immunomodulatory signaling cascade that changed the Ca²⁺ phenotype and downstream transcriptomics. Panel E shows time course traces of different cells (as associated with different code microbeads) under stimulation from the same self- or non-self-antigenic peptide. Phenotypic time courses were observed and quantified for a cell, and subsequently associated with changes in the underlying cell's transcriptomic state via the spectral microbead:sequence linkage.

In addition to monitoring dynamic activation via microscopy, other phenotypic assays have been employed for this assay including monitoring of cytokine secretion from CD4⁺ stimulated T-cells (stimulated non-specifically with drug cocktails e.g., PMA Ionomycin, or specifically in co-culture). For example, cytokines (e.g., INF-gamma, IL2, IL4, IL6, etc.) can be profiled per cell to generate a functional phenotype per cell of cytokines secreted per time point, Additional phenotypic assays may also include dynamic monitoring of protein translocation and movement within a cell using a fluorescent stain or transgenic fluorescent protein coupled to the protein-of-interest.

Phenotypic data is acquired by scanning the entire array via microscopy (FIG. 3, Panel B) in brightfield to locate well boundaries and fiducial marks for later identification of well position as well as characterize cell morphologies and boundaries and the relevant fluorescent wavelengths at sub-cellular resolution for protein or dye tracking at each time point, creating a large-array of data capable of matching well position to cell to dynamic behavior when compiled. Immediately following the acquisition of all cellular phenotypic data, the array is imaged across the lanthanide spectral encoding wavelengths at deep UV excitation as described in Nguyen et al. 2017, supra and brightfield to match well position and fiducial marks with co-encapsulated spectrally encoded beads. After processing these data and deconvolving bead code sets as described in Nguyen et al. 2017, supra., the data set can correlate well position to cellular phenotype per time point to deconvolved unique spectral code per cell. Computationally, image-based data is coupled via a cell-segmentation and tracking algorithm similar to that described in Lane et al, 2017, supra, and Van Valen et al., PLoS Comp. Biol. 12. 11, 2016, where each well position is an object in an ordered array with associated cellular and bead data; wells containing both a cell and bead are considered successful, and only these bead codes are used further in downstream processing. Any bead spectral codes that are identified as non-unique are removed from the set as well as any bead codes associated with multiple cells per well.

Immediately following the collection of cellular phenotypic data and spectral unmixing of each bead's code, cells within the assay are heat-lysed at 95 C for 1 min in a flatbed themocycler. During a brief wait time, cellular-derived transcripts are recruited to the bead surface and bound by poly-adenylation capture sites; this process is isolated per each well. The assay is then disassembled, and the beads containing captured cellular transcripts from a single cell are isolated (see Methods). Beads are processed via SmartSeq-2 modified protocols similar to Picelli et al., Nat. Protocols 9:171, 2014., as described in Macoksco et al. (2015) for reverse transcription, polymerase chain reaction, and next generation library preparation to generate single cell libraries with deterministic barcodes per spectral bead code described as the Unique Spectral Identifier. 300-1,000 beads can be processed for a single library. Capture efficiency is estimated to be 10-30% dependent on well size, consistent with literature estimates of 7-30% in assays such as Mackosco et al., Cell 161:1202-12142, 2015 and Klein et al., Cell 161:1187-1201, 2015, correlating to ˜10,000-100,000 transcripts per cell per bead; available priming and capture sites on the hydrogel bead surface were confirmed orthogonally via qPCR, sequencing, and fluorescent dilution assays to be on the range of 109 sites per bead, suggesting typical diffusion limitations as reducing capture efficiency rather than bead quality. Sequencing of transcriptomic single cell libraries containing the Unique Spectral Identifier processed from spectral beads was conducted via paired-end Illumina sequencing using typical P5/P7 adapters (Picelli et al. 2014, supra,) on a Next-Seq sequencer at high-depth (300M reads/library) per 300-1,000 beads, dependent on depth-requirements of the assay. For inspection of low-expressing genes (e.g., transcription factors, modulatory activation cascades) cells were sequenced above 100,000 reads per cell; typical runs are comparable to Mackosco et al, 2015, supra).

After collecting next generation sequencing data, data is demultiplexed and processed via typical transcriptomic workflows including those described in Mackosco et al. 2015, supm, (STAR algorithm alignment, barocode funneling, read conversion) to convert raw reads to expressed genes within a normalized digital expression matrix; unlike the combinatorial barcodes in Mackosco et al., 2015, barcodes containing our Unique Spectral Identifier were mapped via deterministic matching to a known list given the spectral codes in the assay with allowance for hamming distance mismatches by code, allowing for direct 1:1 mapping of a cell's genetic expression matrix (i.e., list of genes and differential counts by gene) with its unique, individual spectral code ordered by the Unique Spectral Identifier. Subsequently, using a custom algorithm, microscopy data processed by well position in the object data frame containing data from classified cellular phenotypes via time-course microscopy and matched spectral bead codes is coupled to gene expression data from transcriptomic sequencing by mapping the Unique Spectral Identifier deterministically to present bead codes by a simple word recognition and matching algorithm. Mismatches are not tolerated at this stage of analysis, given earlier code assignment and barcode tolerance.

The dataset then contains transcriptomic bead data directly matched to each single cell classified phenotypes via 1:1 linkage with the spectral bead code and identifier linkage. Dependent on the phenotype, causative linkages between resultant single cell phenotype by class of action (similar to Lane et al., 2017, supra) are assigned to underlying bead transcriptome using punative mechanistic regulators of cellular action and activity. For instance, for CD4+ primary or cultured T cell activation phenotypes have been shown to have a diverse set of activator cascades underlying effector functionality and cytokine secretion. Transcriptomic data from this data set is processed and analyzed for differential expression of cytokines and nuclear factors (e.g., interleukins, growth factors, small nuclear modulators, interferon type 1), known targets in the GATA3 and other transcription factor cascades, and specific regulation of stimulatory receptor expression (PD1, OX40, Tim3) compared to non-activated T cells; comparative data is available for epigenomic and transcriptomic linkages in Gate et al., Nature Genet. 50: 1140-1150, 2018), but effector differences on resultant cellular phenotype have not been well-described. Phenotypic effector data on activation and cytokine secretion on the single cell level can classify cells into different effector functionalities (e.g., Th1, Th2, Th17, Treg), but the causative match to transcriptomic data confirms or dispels putative regulators of these canonical phenotypic classification as well as identifies previously unknown genetic pathways underlying phenotypic modulation and regulation. Data sets collected across one experiment (e.g., activated CD4+ T cell) can be compared to other conditions (e.g., non-activated) after batch normalization typical of existing single cell methods.

### Extended Methods

### Solid Phase Support Hydrogel Bead Oligo Conjugation

Oligonucleotide conjugation of a common oligonucleotide (“common sequence”) before attachment of the unique spectral identifier per bead code (as described in the patent text) can be conducted in multiple manners including: (a) direct addition of an acrydite oligonucleotide into the hydrogel matrix during bead polymerization and (b) Michael addition via carboxylation and EDC chemistry. Each of these methods has been successful in creating a common oligonucleotide “linker” in which subsequent Klenow extension PCR takes place to attach the unique spectral identifier. Direct conjugation of the full-length oligonucleotide containing the unique spectral identifier has also been successful.

### (a1) Acrydite Direct Oligonucleotide Hydrogel Incorporation

Spectrally encoded beads were fabricated using lanthanide nanophosphors and PEG-diacrylate hydrogel matrix as described in Nguyen et al. Adv. Opt. Mat. (2017, supra) and Brower et al. JoVE 119 (2017): e55276.). Direct addition of anchor oligonucleotide (“common sequence”) was achieved during synthesis using 10% w/v incorporation of an acrydite-modified oligonucleotide (see furnished sequences) at 10 μM stock in water (IDT, Table 1) with typical UV polymerization protocols as described. The water fraction of the hydrogel mixture is reduced to accommodate this additional volumetric fraction (Nguyen et al. 2017, supra). This oligonucleotide is directly photopolymerized into the PEG-diacrylate (or similar) host matrix during bead generation, and beads can be washed via PBS 1% SDS and 1% Tween exchange protocols as described (Brower et al. 2017, supra). Polymerization time is 3 s at >7 mW/cm² using a spot light source at deep UV 280 nm (Dymax UV-B). Klenow protocols, described below, to extend the single-stranded oligo are performed immediately following hydrogel production.

### (b1) Carboxylation Via Michael Addition

Spectrally encoded beads were fabricated using lanthanide nanophosphors and PEG-diacrylate hydrogel matrix as described in Nguyen et al. and Brower et al. (both supra). Functionalization of the bead surface was achieved following bead fabrication and polymerization through carboxylation of non-crosslinked acrylate groups using typical Michael addition protocols. Beads first were washed with DMF, DCM, and Methanol, then again with DMF. Beads were then incubated in DMF for 15 min to allow bead swelling, followed by addition of 10 molar equivalents (50 mg of hydrogel beads, 10⁹ accessible functional sites per bead, as estimated by next-generation sequencing) of pyridine then carboxy-PEG12-thiol (CT-PEG, Life Technologies). CT-PEG provides a flexible spacer such that bead-bound sequences are highly accessible during transcript hybridization. Beads were then incubated at room temperature overnight with rotation, followed by bead washing in DMSO.

### (b2) Common Sequence Oligonucleotide Conjugation Via EDC

Following functionalization of beads with carboxyl groups as described, addition of single-stranded anchor oligonucleotides (“common sequence”) was achieved via EDC chemistry to attach amine-modified oligonucleotides at 100 μM stock in water (IDT, Table 1). Carboxylated beads were incubated with 75 mM EDC (1-Ethyl-3-(3-dimethylaminopropyl)carbodiimide), 15 mM HOAt (1-Hydroxy-7-azabenzotriazole), and 75 mM DIPEA (N,N-Diisopropylethylamine) for 15 min at room temperature with rotation, followed by addition of this amine-modified oligonucleotide. Beads were incubated at room temperature overnight with rotation, followed by neutralization with 200 mM Tris HCl for 30 min and washing with PBS 0.1% Tween.

### Barcode Selection

Unique spectral identifier barcodes were selected for direct and Klenow extended conjugations via deterministic rational design using Hamming distances for maximal sequencing discrimination of spectral codes. Dependent on the spectral code size, barcodes were selected for Hamming distances of 3-4 nt. For a sample set of 96 spectral codes shown in Tables 2 and 3, barcodes of size 9 nt were rationally designed with a hamming distance of 4 nt for maximal sequencing specificity during next-generation sequencing demultiplexing, an error-prone process. Common algorithms were used for barcode design dependent on code size (Bioconductor, DNA barcodes) and optimized pools for larger code sets were directly adapted from literature (Glenn et al., Plos ONE, 2012: doi:10.1371/journal.pone.0042543).

Klenow extension of Spectral Identifier Oligonucleotides

Klenow reactions for extension of single-stranded DNA were performed on aliquots of 500 beads to extend single-stranded anchor oligonucleotides (“common sequence”) into full-length sequences containing the unique spectral identifier per each bead code; reactions were performed in wells sequestered by bead code. Beads were incubated at 95° C. with shaking with 1×NEB2 buffer (or similar), 0.2 mM dNTPs, and extension template oligonucleotide at 100 μM stock in water to fully denature anchor and extension oligonucleotides. Beads were then cooled to room temperature to allow annealing of extension template. Five units of Klenow enzyme fragment (3′->5′ exo-) were then added to the reaction to catalyze extension of the anchor oligonucleotide into a full-length, extended, double-stranded oligonucleotide. Beads were then heated to 95° C. to release the un-anchored strand from the beads, and washed with hot PBS 0.1% Tween by each well (each well containing a unique code). Exemplary direct and Klenow extension oligonucleotides are shown in Table 2 and 3 respectively.

qPCR Validation of Klenow Extension for Full Length Bead Sequence

Extension to full-length oligonucleotides was verified with standard qPCR methods, using oligonucleotide primers complementary to the 3′ and 5′ ends of the full-length, extended oligonucleotide (IDT, Table 4). An aliquot of functionalized beads was serially diluted; 5 PCR cycles were performed on each sample; 1 μL of supernatant from the bead-based reaction was removed. Each removed sample was combined with 1X iTaq Universal SYBR Green Supermix (or similar) and 375 nM each primer to a 10 μL total reaction. Thirty five cycles of qPCR were performed under manufacturer conditions.

### Hydrosgel and Bead Loading of the Assay

Hydrogel beads conjugated with spectral identifier oligonucleotides were diluted to 50,000 beads/mL and suspended over a dense microwell array, activated to be hydrophilic (microwells 50-100 μm each in width and depth; 100,000 microwells/array) via 30 min treatment in 5% BSA. Beads suspension was derived from pooling all codes from successful qPCR Klenow validation reactions by well (˜96-1,1102 codes pooled dependent on assay) with equal representation from each code by quantitative qPCR DNA content adjustment. Beads were loaded via slow release of 100 μL bead suspension over the microwell array surface. The loading suspension was subsequently spread using a P200 pipette tip for full coverage of the array. Beads were allowed to settle for 5 minutes by gravity. Cell suspension (5,000-2,500,000 cells/mL) was then slowly added to the wetted microwell surface by serial addition of 20 μL of fluid, up to 100 μL, across the surface of the area. Cells were gently spread using a P200 pipette tip and allowed to settle for 5 min by gravity. At this point, the array contains co-encapsulated beads and cells. The microwell device was then gently sealed using a glass slide placed on the wetted microwell surface and securely clamped using a custom acrylic clamping apparatus with set screws.

### Device Operation

Bead codes and co-encapsulated cells were imaged for a cellular functional phenotype as shown in FIG. 3. Bead codes were deconvolved as previously reported in Nguyen et al. (2017, supra). Following cellular and bead imaging via image acquisition of the full microwell array, the array is heated to 95° C. using a flatbed thermocycler (Life Technologies) for 1 minute to lyse cells via heat-lysis (other lysis mechanisms possible). Hybridization of released cellular transcripts to poly-A capture oligonucleotides containing unique spectral identifiers (Tables 3 and 4) on the bead surface proceeds for 10 minutes at room temperature; beads and cellular contents remain sequestered by well during this process. Following hybridization, the clamping apparatus containing the microwell array is moved to a bath containing 100 mL of 2.5% BSA solution (Life Technologies) and is dissembled via release of each set screw. The bath is shaken for 30 s at low speed using a benchtop rocker to release beads from microwells; the bath contents are poured into (2) 50 mL Falcon tubes and spun down at 400 g using a centrifuge to concentrate microwell-derived beads. The supernatant is discarded from each tube, reserving 50 μL containing the recovered spectral bead pellet for transfer to RT-PCR reactions to amplify bead-bound transcripts and prepare for next-generation sequencing. From this point, protocols are consistent with modified SmartSeq 2 (Piccelli et al., Nat. Met. 10 1096-1098, 2013); exact bead-based RT and PCR processing steps are derived from the DropSeq workflow (Mackosco et al., Cell 161:1202-12142, 2015). Next-generation sequencing on final fragments was conducted via NextSeq High Output 500M reads with demultiplexing by unique spectral identifier via a customized sequence processing pipeline; a custom amount of PhiX spike-in (20%) was used to increase sequencing diversity due to deterministic barcode schemes of our method compared to alternative combinatorial techniques (e.g., DropSeq).

### Illustrative Sequences.

TABLE 1
Amine and Acrydite Oligonucleodes (‘common
sequences’). The oligonucleotides can be
further extended via Klenow extension (Table 3)
to contain the unique spectral identifier and
the unique molecular identifiers needed for
embodiments of this assay. Any common sequence
can be used (for purposes of illustration the
sequences provided below are similar to
Mackosco et al. 2015, supra). Common replacements
would include promoter systems (e.g., a T7
promoter system (Klein et al., Cell 163:1187-
1201, 2015)) or diverse common backbones to
increase sequencing diversity.

| Oligonucleotide | Sequence |
|---|---|
| Amine common | /5AmMC6/TTTTTTTAAGCAGTC |
| | GTATCAACGCAGAGTAC |
| | (SEQ ID NO: 1) |
| Acrydite common | /5Acryd/TTTTTTTAAGCAGTG |
| | GTATCAACGCAGAGTAC |
| | (SEQ ID NO: 2) |

TABLE 2
Direct Conjugation Oligonucleotides
(96 spectral barcodes, representative set).
These oligonucleotides contain the common
sequence, representative 9 nucleotide
unique spectral identifier barcode
(hamming distance = 4, USI shown
underlined in first sequence),
unique molecular identifier
(denoted as Ns) and anchored
polyadenylation capture sequence.
The oligonucleotides are directly
conjugated following Michael addition
by code in each reaction vessel
(less preferable than Kienow
extension). SEQ ID NOS = 3-98

| | direct |
|---|---|
| Barcode | conjugation |
| ID | sequence |
| bc1 | AAGCAGTGGTATCAACGCAGAGTACTTAAGGCC |
| | TNNNNNNNNTTTTTTTTTTTTTTTTTTTTTTTT |
| | TTTTTT |
| bc2 | AAGCAGTGGTATCAACGCAGAGTACAGGACATG |
| | TNNNNNNNNTTTTTTTTTTTTTTTTTTTTTTTT |
| | TTTTTT |
| bc3 | AAGCAGTGGTATCAACGCAGAGTACACCAGGCT |
| | TNNNNNNNNTTTTTTTTTTTTTTTTTTTTTTTT |
| | TTTTTT |
| bc4 | AAGCAGTGGTATCAACGCAGAGTACCAGGAATC |
| | GNNNNNNNNTTTTTTTTTTTTTTTTTTTTTTTT |
| | TTTTTT |
| bc5 | AAGCAGTGGTATCAACGCAGAGTACGATCCTTC |
| | GNNNNNNNNTTTTTTTTTTTTTTTTTTTTTTTT |
| | TTTTTT |
| bc6 | AAGCAGTGGTATCAACGCAGAGTACCCGTCATA |
| | TNNNNNNNNTTTTTTTTTTTTTTTTTTTTTTTT |
| | TTTTTT |
| bc7 | AAGCAGTGGTATCAACGCAGAGTACAGTCCACA |
| | ANNNNNNNNTTTTTTTTTTTTTTTTTTTTTTTT |
| | TTTTTT |
| bc 8 | AAGCAGTGGTATCAACGCAGAGTACCATCAGCC |
| | TNNNNNNNNTTTTTTTTTTTTTTTTTTTTTTTT |
| | TTTTTT |
| bc9 | AAGCAGTGGTATCAACGCAGAGTACTTCTATGG |
| | CNNNNNNNNTTTTTTTTTTTTTTTTTTTTTTTT |
| | TTTTTT |
| bc10 | AAGCAGTGGTATCAACGCAGAGTACCCTCGTAA |
| | GNNNNNNNNTTTTTTTTTTTTTTTTTTTTTTTT |
| | TTTTTT |
| bc11 | AAGCAGTGGTATCAACGCAGAGTACTCGCAATT |
| | CNNNNNNNNTTTTTTTTTTTTTTTTTTTTTTTT |
| | TTTTTT |
| bc12 | AAGCAGTGGTATCAACGCAGAGTACCTTGGTGC |
| | ANNNNNNNNTTTTTTTTTTTTTTTTTTTTTTTT |
| | TTTTTT |
| bc13 | AAGCAGTGGTATCAACGCAGAGTACAGAAGCTT |
| | CNNNNNNNNTTTTTTTTTTTTTTTTTTTTTTTT |
| | TTTTTT |
| bc14 | AAGCAGTGGTATCAACGCAGAGTACTGTTGGAG |
| | GNNNNNNNNTTTTTTTTTTTTTTTTTTTTTTTT |
| | TTTTTT |
| bc15 | AAGCAGTGGTATCAACGCAGAGTACCCACATTC |
| | GNNNNNNNNTTTTTTTTTTTTTTTTTTTTTTTT |
| | TTTTTT |
| bc16 | AAGCAGTGGTATCAACGCAGAGTACCAATGGTC |
| | GNNNNNNNNTTTTTTTTTTTTTTTTTTTTTTTT |
| | TTTTTT |
| bc17 | AAGCAGTGGTATCAACGCAGAGTACATATGCGG |
| | TNNNNNNNNTTTTTTTTTTTTTTTTTTTTTTTT |
| | TTTTTT |
| bc18 | AAGCAGTGGTATCAACGCAGAGTACCTGTAACA |
| | GNNNNNNNNTTTTTTTTTTTTTTTTTTTTTTTT |
| | TTTTTT |
| bc19 | AAGCAGTGGTATCAACGCAGAGTACTTCCTCAA |
| | GNNNNNNNNTTTTTTTTTTTTTTTTTTTTTTTT |
| | TTTTTT |
| bc20 | AAGCAGTGGTATCAACGCAGAGTACCAAGTTGG |
| | TNNNNNNNNTTTTTTTTTTTTTTTTTTTTTTTT |
| | TTTTTT |
| bc21 | AAGCAGTGGTATCAACGCAGAGTACTCGTGAGG |
| | TNNNNNNNNTTTTTTTTTTTTTTTTTTTTTTTT |
| | TTTTTT |
| bc22 | AAGCAGTGGTATCAACGCAGAGTACACTTCGGA |
| | ANNNNNNNNTTTTTTTTTTTTTTTTTTTTTTTT |
| | TTTTTT |
| bc23 | AAGCAGTGGTATCAACGCAGAGTACACTGCCTG |
| | TNNNNNNNNTTTTTTTTTTTTTTTTTTTTTTTT |
| | TTTTTT |
| bc24 | AAGCAGTGGTATCAACGCAGAGTACGAAGTCTC |
| | GNNNNNNNNTTTTTTTTTTTTTTTTTTTTTTTT |
| | TTTTTT |
| bc25 | AAGCAGTGGTATCAACGCAGAGTACTCCTGGAA |
| | TNNNNNNNNTTTTTTTTTTTTTTTTTTTTTTTT |
| | TTTTTT |
| bc26 | AAGCAGTGGTATCAACGCAGAGTACTCTCTTCG |
| | GNNNNNNNNTTTTTTTTTTTTTTTTTTTTTTTT |
| | TTTTTT |
| bc27 | AAGCAGTGGTATCAACGCAGAGTACAGCCATGG |
| | ANNNNNNNNTTTTTTTTTTTTTTTTTTTTTTTT |
| | TTTTTT |
| bc28 | AAGCAGTGGTATCAACGCAGAGTACCTCTGTTG |
| | TNNNNNNNNTTTTTTTTTTTTTTTTTTTTTTTT |
| | TTTTTT |
| bc29 | AAGCAGTGGTATCAACGCAGAGTACGCTACACA |
| | TNNNNNNNNTTTTTTTTTTTTTTTTTTTTTTTT |
| | TTTTTT |
| bc30 | AAGCAGTGGTATCAACGCAGAGTACCGTATCCA |
| | TNNNNNNNNTTTTTTTTTTTTTTTTTTTTTTTT |
| | TTTTTT |
| bc31 | AAGCAGTGGTATCAACGCAGAGTACTGTCCTTG |
| | TNNNNNNNNTTTTTTTTTTTTTTTTTTTTTTTT |
| | TTTTTT |
| bc32 | AAGCAGTGGTATCAACGCAGAGTACTTGGCACG |
| | ANNNNNNNNTTTTTTTTTTTTTTTTTTTTTTTT |
| | TTTTTT |
| bc33 | AAGCAGTGGTATCAACGCAGAGTACGGCGAACT |
| | TNNNNNNNNTTTTTTTTTTTTTTTTTTTTTTTT |
| | TTTTTT |
| bc34 | AAGCAGTGGTATCAACGCAGAGTACGTATGTTC |
| | CNNNNNNNNTTTTTTTTTTTTTTTTTTTTTTTT |
| | TTTTTT |
| bc35 | AAGCAGTGGTATCAACGCAGAGTACTTAGGACA |
| | CNNNNNNNNTTTTTTTTTTTTTTTTTTTTTTTT |
| | TTTTTT |
| bc36 | AAGCAGTGGTATCAACGCAGAGTACTCGGACCT |
| | TNNNNNNNNTTTTTTTTTTTTTTTTTTTTTTTT |
| | TTTTTT |
| bc37 | AAGCAGTGGTATCAACGCAGAGTACGCATCTTG |
| | TNNNNNNNNTTTTTTTTTTTTTTTTTTTTTTTT |
| | TTTTTT |
| bc38 | AAGCAGTGGTATCAACGCAGAGTACCTTAGGAG |
| | TNNNNNNNNTTTTTTTTTTTTTTTTTTTTTTTT |
| | TTTTTT |
| bc39 | AAGCAGTGGTATCAACGCAGAGTACCTCAGCTA |
| | CNNNNNNNNTTTTTTTTTTTTTTTTTTTTTTTT |
| | TTTTTT |
| bc40 | AAGCAGTGGTATCAACGCAGAGTACGCAGGTAT |
| | TNNNNNNNNTTTTTTTTTTTTTTTTTTTTTTTT |
| | TTTTTT |
| bc41 | AAGCAGTGGTATCAACGCAGAGTACTGCGTAAG |
| | CNNNNNNNNTTTTTTTTTTTTTTTTTTTTTTTT |
| | TTTTTT |
| bc42 | AAGCAGTGGTATCAACGCAGAGTACATTCCTCT |
| | CNNNNNNNNTTTTTTTTTTTTTTTTTTTTTTTT |
| | TTTTTT |
| bc43 | AAGCAGTGGTATCAACGCAGAGTACTGGAACGT |
| | CNNNNNNNNTTTTTTTTTTTTTTTTTTTTTTTT |
| | TTTTTT |
| bc44 | AAGCAGTGGTATCAACGCAGAGTACGGATTCCT |
| | TNNNNNNNNTTTTTTTTTTTTTTTTTTTTTTTT |
| | TTTTTT |
| bc45 | AAGCAGTGGTATCAACGCAGAGTACCTACGCCT |
| | TNNNNNNNNTTTTTTTTTTTTTTTTTTTTTTTT |
| | TTTTTT |
| bc46 | AAGCAGTGGTATCAACGCAGAGTACTGAGGCGT |
| | TNNNNNNNNTTTTTTTTTTTTTTTTTTTTTTTT |
| | TTTTTT |
| bc47 | AAGCAGTGGTATCAACGCAGAGTACTAGACGCA |
| | CNNNNNNNNTTTTTTTTTTTTTTTTTTTTTTTT |
| | TTTTTT |
| bc48 | AAGCAGTGGTATCAACGCAGAGTACTGGTCCGA |
| | ANNNNNNNNTTTTTTTTTTTTTTTTTTTTTTTT |
| | TTTTTT |
| bc49 | AAGCAGTGGTATCAACGCAGAGTACTTCACCAT |
| | CNNNNNNNNTTTTTTTTTTTTTTTTTTTTTTTT |
| | TTTTTT |
| bc50 | AAGCAGTGGTATCAACGCAGAGTACGTCGATAA |
| | CNNNNNNNNTTTTTTTTTTTTTTTTTTTTTTTT |
| | TTTTTT |
| bc51 | AAGCAGTGGTATCAACGCAGAGTACCTGAGATG |
| | GNNNNNNNNTTTTTTTTTTTTTTTTTTTTTTTT |
| | TTTTTT |
| bc52 | AAGCAGTGGTATCAACGCAGAGTACTTAGCCTG |
| | CNNNNNNNNTTTTTTTTTTTTTTTTTTTTTTTT |
| | TTTTTT |
| bc53 | AAGCAGTGGTATCAACGCAGAGTACCAGTTGTA |
| | CNNNNNNNNTTTTTTTTTTTTTTTTTTTTTTTT |
| | TTTTTT |
| bc54 | AAGCAGTGGTATCAACGCAGAGTACCCAACTGA |
| | ANNNNNNNNTTTTTTTTTTTTTTTTTTTTTTTT |
| | TTTTTT |
| bc55 | AAGCAGTGGTATCAACGCAGAGTACCTGACCAA |
| | TNNNNNNNNTTTTTTTTTTTTTTTTTTTTTTTT |
| | TTTTTT |
| bc56 | AAGCAGTGGTATCAACGCAGAGTACCGATAGCT |
| | GNNNNNNNNTTTTTTTTTTTTTTTTTTTTTTTT |
| | TTTTTT |
| bc57 | AAGCAGTGGTATCAACGCAGAGTACGATAAGGA |
| | GNNNNNNNNTTTTTTTTTTTTTTTTTTTTTTTT |
| | TTTTTT |
| bc58 | AAGCAGTGGTATCAACGCAGAGTACACAGCGAG |
| | ANNNNNNNNTTTTTTTTTTTTTTTTTTTTTTTT |
| | TTTTTT |
| bc59 | AAGCAGTGGTATCAACGCAGAGTACACTCATTG |
| | CNNNNNNNNTTTTTTTTTTTTTTTTTTTTTTTT |
| | TTTTTT |
| bc60 | AAGCAGTGGTATCAACGCAGAGTACAAGAGTGC |
| | GNNNNNNNNTTTTTTTTTTTTTTTTTTTTTTTT |
| | TTTTTT |
| bc61 | AAGCAGTGGTATCAACGCAGAGTACTAACCGTT |
| | CNNNNNNNNTTTTTTTTTTTTTTTTTTTTTTTT |
| | TTTTTT |
| bc62 | AAGCAGTGGTATCAACGCAGAGTACGTTACAAG |
| | CNNNNNNNNTTTTTTTTTTTTTTTTTTTTTTTT |
| | TTTTTT |
| bc63 | AAGCAGTGGTATCAACGCAGAGTACCAAGCGAT |
| | TNNNNNNNNTTTTTTTTTTTTTTTTTTTTTTTT |
| | TTTTTT |
| bc64 | AAGCAGTGGTATCAACGCAGAGTACGGAATTGC |
| | CNNNNNNNNTTTTTTTTTTTTTTTTTTTTTTTT |
| | TTTTTT |
| bc65 | AAGCAGTGGTATCAACGCAGAGTACGATCGTCT |
| | TNNNNNNNNTTTTTTTTTTTTTTTTTTTTTTTT |
| | TTTTTT |
| bc66 | AAGCAGTGGTATCAACGCAGAGTACTAGCCTAT |
| | GNNNNNNNNTTTTTTTTTTTTTTTTTTTTTTTT |
| | TTTTTT |
| bc67 | AAGCAGTGGTATCAACGCAGAGTACACACGGAC |
| | TNNNNNNNNTTTTTTTTTTTTTTTTTTTTTTTT |
| | TTTTTT |
| bc68 | AAGCAGTGGTATCAACGCAGAGTACTACAGAGT |
| | CNNNNNNNNTTTTTTTTTTTTTTTTTTTTTTTT |
| | TTTTTT |
| bc69 | AAGCAGTGGTATCAACGCAGAGTACTTCGAGGT |
| | TNNNNNNNNTTTTTTTTTTTTTTTTTTTTTTTT |
| | TTTTTT |
| bc70 | AAGCAGTGGTATCAACGCAGAGTACATTGGCCA |
| | TNNNNNNNNTTTTTTTTTTTTTTTTTTTTTTTT |
| | TTTTTT |
| bc71 | AAGCAGTGGTATCAACGCAGAGTACCATCTCGC |
| | ANNNNNNNNTTTTTTTTTTTTTTTTTTTTTTTT |
| | TTTTTT |
| bc72 | AAGCAGTGGTATCAACGCAGAGTACCCGAATTG |
| | TNNNNNNNNTTTTTTTTTTTTTTTTTTTTTTTT |
| | TTTTTT |
| bc73 | AAGCAGTGGTATCAACGCAGAGTACCCTTGTGT |
| | TNNNNNNNNTTTTTTTTTTTTTTTTTTTTTTTT |
| | TTTTTT |
| bc74 | AAGCAGTGGTATCAACGCAGAGTACGGCCATTA |
| | TNNNNNNNNTTTTTTTTTTTTTTTTTTTTTTTT |
| | TTTTTT |
| bc75 | AAGCAGTGGTATCAACGCAGAGTACCACACTAG |
| | GNNNNNNNNTTTTTTTTTTTTTTTTTTTTTTTT |
| | TTTTTT |
| bc76 | AAGCAGTGGTATCAACGCAGAGTACTGGTTGTC |
| | GNNNNNNNNTTTTTTTTTTTTTTTTTTTTTTTT |
| | TTTTTT |
| bc77 | AAGCAGTGGTATCAACGCAGAGTACCACCATGT |
| | GNNNNNNNNTTTTTTTTTTTTTTTTTTTTTTTT |
| | TTTTTT |
| bc78 | AAGCAGTGGTATCAACGCAGAGTACGGCTTGGA |
| | TNNNNNNNNTTTTTTTTTTTTTTTTTTTTTTTT |
| | TTTTTT |
| bc79 | AAGCAGTGGTATCAACGCAGAGTACACGTCAAC |
| | CNNNNNNNNTTTTTTTTTTTTTTTTTTTTTTTT |
| | TTTTTT |
| bc80 | AAGCAGTGGTATCAACGCAGAGTACACCTTAAG |
| | GNNNNNNNNTTTTTTTTTTTTTTTTTTTTTTTT |
| | TTTTTT |
| bc81 | AAGCAGTGGTATCAACGCAGAGTACCGGTCTAC |
| | ANNNNNNNNTTTTTTTTTTTTTTTTTTTTTTTT |
| | TTTTTT |
| bc82 | AAGCAGTGGTATCAACGCAGAGTACCTTCTTAC |
| | CNNNNNNNNTTTTTTTTTTTTTTTTTTTTTTTT |
| | TTTTTT |
| bc83 | AAGCAGTGGTATCAACGCAGAGTACATGCTGTT |
| | CNNNNNNNNTTTTTTTTTTTTTTTTTTTTTTTT |
| | TTTTTT |
| bc84 | AAGCAGTGGTATCAACGCAGAGTACTGCTACAT |
| | GNNNNNNNNTTTTTTTTTTTTTTTTTTTTTTTT |
| | TTTTTT |
| bc85 | AAGCAGTGGTATCAACGCAGAGTACAGAACCGC |
| | TNNNNNNNNTTTTTTTTTTTTTTTTTTTTTTTT |
| | TTTTTT |
| bc86 | AAGCAGTGGTATCAACGCAGAGTACTTGTCAGT |
| | CNNNNNNNNTTTTTTTTTTTTTTTTTTTTTTTT |
| | TTTTTT |
| bc87 | AAGCAGTGGTATCAACGCAGAGfACATCCAAGC |
| | CNNNNNNNNTTTTTTTTTTTTTTTTTTTTTTTT |
| | TTTTTT |
| bc88 | AAGCAGTGGTATCAACGCAGAGTACCTAGTCAC |
| | TNNNNNNNNTTTTTTTTTTTTTTTTTTTTTTTT |
| | TTTTTT |
| bc89 | AAGCAGTGGTATCAACGCAGAGTACCAGTGGCT |
| | TNNNNNNNNTTTTTTTTTTTTTTTTTTTTTTTT |
| | TTTTTT |
| bc90 | AAGCAGTGGTATCAACGCAGAGTACTGGCACAC |
| | TNNNNNNNNTTTTTTTTTTTTTTTTTTTTTTTT |
| | TTTTTT |
| bc91 | AAGCAGTGGTATCAACGCAGAGTACCGGTAAGT |
| | TNNNNNNNNTTTTTTTTTTTTTTTTTTTTTTTT |
| | TTTTTT |
| bc92 | AAGCAGTGGTATCAACGCAGAGTACGGTTGATT |
| | CNNNNNNNNTTTTTTTTTTTTTTTTTTTTTTTT |
| | TTTTTT |
| bc93 | AAGCAGTGGTATCAACGCAGAGTACCATCTACT |
| | GNNNNNNNNTTTTTTTTTTTTTTTTTTTTTTTT |
| | TTTTTT |
| bc94 | AAGCAGTGGTATCAACGCAGAGTACCTAGTGGA |
| | GNNNNNNNNTTTTTTTTTTTTTTTTTTTTTTTT |
| | TTTTTT |
| bc95 | AAGCAGTGGTATCAACGCAGAGTACTCACTCTC |
| | CNNNNNNNNTTTTTTTTTTTTTTTTTTTTTTTT |
| | TTTTTT |
| bc96 | AAGCAGTGGTATCAACGCAGAGTACCCACAAGA |
| | CNNNNNNNNTTTTTTTTTTTTTTTTTTTTTTTT |
| | TTTTTT |

TABLE 3
Klenow Extension Oligonucleotides (96
spectral barcodes, representative set).
These oligonucleotides contain the
common sequence, representative 9
nucleotide unique spectral identifier
barcode (hamming distance = 4, USI
shown in highlight on first sequence),
unique molecular identifier (denoted as
Ns) and anchored polyadenylation capture
sequence. These oligonucleotides are
conjugated by code after the addition
of the common sequence via Klenow PCR
based extension in a well-based format.
SEQ ID NOS = 99-194

| Barocde | |
|---|---|
| ID | Klenow extension sequence |
| bc1 | NBAAAAAAAAAAAAAAAAAAAAAAA |
| | AAAAAAANNNNNNNNAGCCCTTAAG |
| | TACTCTGCGTTGATACCACTGCTT |
| bc2 | NBAAAAAAAAAAAAAAAAAAAAAAA |
| | AAAAAAANNNNNNNNACATGTCCTG |
| | TACTCTGCGTTGATACCACTGCTT |
| bc3 | NBAAAAAAAAAAAAAAAAAAAAAAA |
| | AAAAAAANNNNNNNNAAGCCTGGTG |
| | TACTCTGCGTTGATACCACTGCTT |
| bc4 | NBAAAAAAAAAAAAAAAAAAAAAAA |
| | AAAAAAANNNNNNNNCGATTCCTGG |
| | TACTCTGCGTTGATACCACTGCTT |
| bc5 | NBAAAAAAAAAAAAAAAAAAAAAAA |
| | AAAAAAANNNNNNNNCGAAGGATCG |
| | TACTCTGCGTTGATACCACTGCTT |
| bc6 | NBAAAAAAAAAAAAAAAAAAAAAAA |
| | AAAAAAANNNNNNNNATATGACGGG |
| | TACTCTGCGTTGATACCACTGCTT |
| bc7 | NBAAAAAAAAAAAAAAAAAAAAAAA |
| | AAAAAAANNNNNNNNTTGTGGACTG |
| | TACTCTGCGTTGATACCACTGCTT |
| bc8 | NBAAAAAAAAAAAAAAAAAAAAAAA |
| | AAAAAAANNNNNNNNAGGCTGATGG |
| | TACTCTGCGTTGATACCACTGCTT |
| bc9 | NBAAAAAAAAAAAAAAAAAAAAAAA |
| | AAAAAAANNNNNNNNGCCATAGAAG |
| | TACTCTGCGTTGATACCACTGCTT |
| bc10 | NBAAAAAAAAAAAAAAAAAAAAAAA |
| | AAAAAAANNNNNNNNCTTACGAGGG |
| | TACTCTGCGTTGATACCACTGCTT |
| bcll | NBAAAAAAAAAAAAAAAAAAAAAAA |
| | AAAAAAANNNNNNNNGAATTGCGAG |
| | TACTCTGCGTTGATACCACTGCTT |
| bcl2 | NBAAAAAAAAAAAAAAAAAAAAAAA |
| | AAAAAAANNNNNNNNTGCACCAAGG |
| | TACTCTGCGTTGATACCACTGCTT |
| bc13 | NBAAAAAAAAAAAAAAAAAAAAAAA |
| | AAAAAAANNNNNNNNGAAGCTTCTG |
| | TACTCTGCGTTGATACCACTGCTT |
| bc14 | NBAAAAAAAAAAAAAAAAAAAAAAA |
| | AAAAAAANNNNNNNNCCTCCAACAG |
| | TACTCTGCGTTGATACCACTGCTT |
| bc15 | NBAAAAAAAAAAAAAAAAAAAAAAA |
| | AAAAAAANNNNNNNNCGAATGTGGG |
| | TACTCTGCGTTGATACCACTGCTT |
| bc16 | NBAAAAAAAAAAAAAAAAAAAAAAA |
| | AAAAAAANNNNNNNNCGACCATTGG |
| | TACTCTGCGTTGATACCACTGCTT |
| bc17 | NBAAAAAAAAAAAAAAAAAAAAAAA |
| | AAAAAAANNNNNNNNACCGCATATG |
| | TACTCTGCGTTGATACCACTGCTT |
| bc18 | NBAAAAAAAAAAAAAAAAAAAAAAA |
| | AAAAAAANNNNNNNNCTGTTACAGG |
| | TACTCTGCGTTGATACCACTGCTT |
| bc19 | NBAAAAAAAAAAAAAAAAAAAAAAA |
| | AAAAAAANNNNNNNNCTTGAGGAAG |
| | TACTCTGCGTTGATACCACTCCTT |
| bc20 | NBAAAAAAAAAAAAAAAAAAAAAAA |
| | AAAAAAANNNNNNNNACCAACTTGG |
| | TACTCTGCGTTGATACCACTGCTT |
| bc21 | NBAAAAAAAAAAAAAAAAAAAAAAA |
| | AAAAAAANNNNNNNNACCTCACGAG |
| | TACTCTGCGTTGATACCACTGCTT |
| bc22 | NBAAAAAAAAAAAAAAAAAAAAAAA |
| | AAAAAAANNNNNNNNTTCCGAAGTG |
| | TACTCTGCGTTGATACCACTGCTT |
| bc23 | NBAAAAAAAAAAAAAAAAAAAAAA |
| | AAAAAAAANNNNNNNNACAGGCAGT |
| | GTACTCTGCGTTGATACCACTGCTT |
| bc24 | NBAAAAAAAAAAAAAAAAAAAAAAA |
| | AAAAAAANNNNNNNNCGAGACTTCG |
| | TACTCTGCGTTGATACCACTGCTT |
| bc25 | NBAAAAAAAAAAAAAAAAAAAAAAA |
| | AAAAAAANNNNNNNNATTCCAGGAG |
| | TACTCTGCGTTGATACCACTGCTT |
| bc26 | NBAAAAAAAAAAAAAAAAAAAAAAA |
| | AAAAAAANNNNNNNNCCGAAGAGAG |
| | TACTCTGCGTTGATACCACTGCTT |
| bc27 | NBAAAAAAAAAAAAAAAAAAAAAAA |
| | AAAAAAANNNNNNNNTCCATGGCTG |
| | TACTCTGCGTTGATACCACTGCTT |
| bc28 | NBAAAAAAAAAAAAAAAAAAAAAAA |
| | AAAAAAANNNNNNNNACAACAGAGG |
| | TACTCTGCGTTGATACCACTGCTT |
| bc29 | NBAAAAAAAAAAAAAAAAAAAAAAA |
| | AAAAAAANNNNNNNNATGTGTAGCG |
| | TACTCTGCGTTGATACCACTGCTT |
| bc30 | NBAAAAAAAAAAAAAAAAAAAAAAA |
| | AAAAAAANNNNNNNNATGGATACGG |
| | TACTCTGCGTTGATACCACTGCTT |
| bc31 | NBAAAAAAAAAAAAAAAAAAAAAAA |
| | AAAAAAANNNNNNNNACAAGGACAG |
| | TACTCTGCGTTGATACCACTGCTT |
| bc32 | NBAAAAAAAAAAAAAAAAAAAAAAA |
| | AAAAAAANNNNNNNNTCGTGCCAAG |
| | TACTCTGCGTTGATACCACTGCTT |
| bc33 | NBAAAAAAAAAAAAAAAAAAAAAAA |
| | AAAAAAANNNNNNNNAAGTTCGCCG |
| | TACTCTGCGTTGATACCACTGCTT |
| bc34 | NBAAAAAAAAAAAAAAAAAAAAAAA |
| | AAAAAAANNNNNNNNGGAACATACG |
| | TACTCTGCGTTGATACCACTGCTT |
| bc35 | NBAAAAAAAAAAAAAAAAAAAAAAA |
| | AAAAAAANNNNNNNNGTGTCCTAAG |
| | TACTCTGCGTTGATACCACTGCTT |
| bc36 | NBAAAAAAAAAAAAAAAAAAAAAAA |
| | AAAAAAANNNNNNNNAAGGTCCGAG |
| | TACTCTGCGTTGATACCACTGCTT |
| bc37 | NBAAAAAAAAAAAAAAAAAAAAAAA |
| | AAAAAAANNNNNNNNACAAGATGCG |
| | TACTCTGCGTTGATACCACTGCTT |
| bc38 | NBAAAAAAAAAAAAAAAAAAAAAAA |
| | AAAAAAANNNNNNNNACTCCTAAGG |
| | TACTCTGCGTTGATACCACTGCTT |
| bc39 | NBAAAAAAAAAAAAAAAAAAAAAAA |
| | AAAAAAANNNNNNNNGTAGCTGAGG |
| | TACTCTGCGTTGATACCACTGCTT |
| bc40 | NBAAAAAAAAAAAAAAAAAAAAAAA |
| | AAAAAAANNNNNNNNAATACCTGCG |
| | TACTCTGCGTTGATACCACTGCTT |
| bc41 | NBAAAAAAAAAAAAAAAAAAAAAAA |
| | AAAAAAANNNNNNNNGCTTACGCAG |
| | TACTCTGCGTTGATACCACTGCTT |
| bc42 | NBAAAAAAAAAAAAAAAAAAAAAAA |
| | AAAAAAANNNNNNNNGAGAGGAATG |
| | TACTCTGCGTTGATACCACTGCTT |
| bc43 | NBAAAAAAAAAAAAAAAAAAAAAAA |
| | AAAAAAANNNNNNNNCACGTTCCAC |
| | TACTCTGCGTTGATACCACTGCTT |
| bc44 | NBAAAAAAAAAAAAAAAAAAAAAAA |
| | AAAAAAANNNNNNNNAAGGAATCCG |
| | TACTCTGCGTTGATACCACTGCTT |
| bc45 | NBAAAAAAAAAAAAAAAAAAAAAAA |
| | AAAAAAANNNNNNNNAAGGCGTAGG |
| | TACTCTGCGTTGATACCACTGCTT |
| bc46 | NBAAAAAAAAAAAAAAAAAAAAAAA |
| | AAAAAAANNNNNNNNAACGCCTCAG |
| | TACTCTGCGTTGATACCACTGCTT |
| bc47 | NBAAAAAAAAAAAAAAAAAAAAAAA |
| | AAAAAAANNNNNNNNGTGCGTCTAG |
| | TACTCTGCGTTGATACCACTGCTT |
| bc48 | NBAAAAAAAAAAAAAAAAAAAAAAA |
| | AAAAAAANNNNNNNNTTCGGACCAG |
| | TACTCTGCGTTGATACCACTGCTT |
| bc49 | NBAAAAAAAAAAAAAAAAAAAAAAA |
| | AAAAAAANNNNNNNNGATGGTGAAG |
| | TACTCTGCGTTGATACCACTGCTT |
| bc50 | NBAAAAAAAAAAAAAAAAAAAAAAA |
| | AAAAAAANNNNNNNNGTTATCGACG |
| | TACTCTGCGTTGATACCACTGCTT |
| bc51 | NBAAAAAAAAAAAAAAAAAAAAAAA |
| | AAAAAAANNNNNNNNCCATCTCAGG |
| | TACTCTGCGTTGATACCACTGCTT |
| bc52 | NBAAAAAAAAAAAAAAAAAAAAAAA |
| | AAAAAAANNNNNNNNGCAGGCTAAG |
| | TACTCTGCGTTGATACCACTGCTT |
| bc53 | NBAAAAAAAAAAAAAAAAAAAAAAA |
| | AAAAAAANNNNNNNNGTACAACTGG |
| | TACTCTGCGTTGATACCACTGCTT |
| bc54 | NBAAAAAAAAAAAAAAAAAAAAAAA |
| | AAAAAAANNNNNNNNTTCAGTTGGG |
| | TACTCTGCGTTGATACCACTGCTT |
| bc55 | NBAAAAAAAAAAAAAAAAAAAAAAA |
| | AAAAAAANNNNNNNNATTGGTCAGG |
| | TACTCTGCGTTGATACCACTGCTT |
| bc56 | NBAAAAAAAAAAAAAAAAAAAAAAA |
| | AAAAAAANNNNNNNNCAGCTATCGG |
| | TACTCTGCGTTGATACCACTGCTT |
| bc57 | NBAAAAAAAAAAAAAAAAAAAAAAA |
| | AAAAAAANNNNNNNNCTCCTTATCG |
| | TACTCTGCGTTGATACCACTGCTT |
| bc58 | NBAAAAAAAAAAAAAAAAAAAAAAA |
| | AAAAAAANNNNNNNNTCTCGCTGTG |
| | TACTCTGCGTTGATACCACTGCTT |
| bc59 | NBAAAAAAAAAAAAAAAAAAAAAAA |
| | AAAAAAANNNNNNNNGCAATGAGTG |
| | TACTCTGCGTTGATACCACTGCTT |
| bc60 | NBAAAAAAAAAAAAAAAAAAAAAAA |
| | AAAAAAANNNNNNNNCGCACTCTTG |
| | TACTCTGCGTTGATACCACTGCTT |
| bc61 | NBAAAAAAAAAAAAAAAAAAAAAAA |
| | AAAAAAANNNNNNNNGAACGGTTAG |
| | TACTCTGCGTTGATACCACTGCTT |
| bc62 | NBAAAAAAAAAAAAAAAAAAAAAAA |
| | AAAAAAANNNNNNNNGCTTGTAACG |
| | TACTCTGCGTTGATACCACTGCTT |
| bc63 | NBAAAAAAAAAAAAAAAAAAAAAAA |
| | AAAAAAANNNNNNNNAATCGCTTGG |
| | TACTCTGCGTTGATACCACTGCTT |
| bc64 | NBAAAAAAAAAAAAAAAAAAAAAAA |
| | AAAAAAANNNNNNNNGGCAATTCCG |
| | TACTCTGCGTTGATACCACTGCTT |
| bc65 | NBAAAAAAAAAAAAAAAAAAAAAAA |
| | AAAAAAANNNNNNNNAAGACGATCG |
| | TACTCTGCGTTGATACCACTGCTT |
| bc66 | NBAAAAAAAAAAAAAAAAAAAAAAA |
| | AAAAAAANNNNNNNNCATAGGCTAG |
| | TACTCTGCGTTGATACCACTGCTT |
| bc67 | NBAAAAAAAAAAAAAAAAAAAAAAA |
| | AAAAAAANNNNNNNNAGTCCGTGTG |
| | TACTCTGCGTTGATACCACTGCTT |
| bc68 | NBAAAAAAAAAAAAAAAAAAAAAAA |
| | AAAAAAANNNNNNNNGACTCTGTAC |
| | TACTCTGCGTTGATACCACTGCTT |
| bc69 | NBAAAAAAAAAAAAAAAAAAAAAAA |
| | AAAAAAANNNNNNNNAACCTCGAAG |
| | TACTCTGCGTTGATACCACTGCTT |
| bc70 | NBAAAAAAAAAAAAAAAAAAAAAAA |
| | AAAAAAANNNNNNNNATGGCCAATG |
| | TACTCTGCGTTGATACCACTGCTT |
| bc71 | NBAAAAAAAAAAAAAAAAAAAAAAA |
| | AAAAAAANNNNNNNNTGCGAGATGG |
| | TACTCTGCGTTGATACCACTGCTT |
| bc72 | NBAAAAAAAAAAAAAAAAAAAAAAA |
| | AAAAAAANNNNNNNNACAATTCGGG |
| | TACTCTGCGTTGATACCACTGCTT |
| bc73 | NBAAAAAAAAAAAAAAAAAAAAAAA |
| | AAAAAAANNNNNNNNAACACAAGGG |
| | TACTCTGCGTTGATACCACTGCTT |
| bc74 | NBAAAAAAAAAAAAAAAAAAAAAAA |
| | AAAAAAANNNNNNNNATAATGGCCG |
| | TACTCTGCGTTGATACCACTGCTT |
| bc75 | NBAAAAAAAAAAAAAAAAAAAAAAA |
| | AAAAAAANNNNNNNNCCTAGTGTGG |
| | TACTCTGCGTTGATACCACTGCTT |
| bc76 | NBAAAAAAAAAAAAAAAAAAAAAA |
| | AAAAAAAANNNNNNNNCGACAACCA |
| | GTACTCTGCGTTGATACCACTGCTT |
| bc77 | NBAAAAAAAAAAAAAAAAAAAAAAA |
| | AAAAAAANNNNNNNNCACATGGTGG |
| | TACTCTGCGTTGATACCACTGCTT |
| bc78 | NBAAAAAAAAAAAAAAAAAAAAAAA |
| | AAAAAAANNNNNNNNATCCAAGCCG |
| | TACTCTGCGTTGATACCACTGCTT |
| bc79 | NBAAAAAAAAAAAAAAAAAAAAAAA |
| | AAAAAAANNNNNNNNGGTTGACGTG |
| | TACTCTGCGTTGATACCACTGCTT |
| bc80 | NBAAAAAAAAAAAAAAAAAAAAAAA |
| | AAAAAAANNNNNNNNCCTTAAGGTG |
| | TACTCTGCGTTGATACCACTGCTT |
| bc81 | NBAAAAAAAAAAAAAAAAAAAAAAA |
| | AAAAAAANNNNNNNNTGTAGACCGG |
| | TACTCTGCGTTGATACCACTGCTT |
| bc82 | NBAAAAAAAAAAAAAAAAAAAAAAA |
| | AAAAAAANNNNNNNNGGTAAGAAGG |
| | TACTCTGCGTTGATACCACTGCTT |
| bc83 | NBAAAAAAAAAAAAAAAAAAAAAAA |
| | AAAAAAANNNNNNNNGAACAGCATG |
| | TACTCTGCGTTGATACCACTGCTT |
| bc84 | NBAAAAAAAAAAAAAAAAAAAAAAA |
| | AAAAAAANNNNNNNNCATGTAGCAC |
| | TACTCTGCGTTGATACCACTGCTT |
| bc85 | NBAAAAAAAAAAAAAAAAAAAAAAA |
| | AAAAAAANNNNNNNNAGCGGTTCTG |
| | TACTCTGCGTTGATACCACTGCTT |
| bc86 | NBAAAAAAAAAAAAAAAAAAAAAAA |
| | AAAAAAANNNNNNNNGACTGACAAG |
| | TACTCTGCGTTGATACCACTGCTT |
| bc87 | NBAAAAAAAAAAAAAAAAAAAAAAA |
| | AAAAAAANNNNNNNNGGCTTGGATG |
| | TACTCTGCGTTGATACCACTGCTT |
| bc88 | NBAAAAAAAAAAAAAAAAAAAAAAA |
| | AAAAAAANNNNNNNNAGTGACTAGG |
| | TACTCTGCGTTGATACCACTGCTT |
| bc89 | NBAAAAAAAAAAAAAAAAAAAAAAA |
| | AAAAAAANNNNNNNNAAGCCACTGG |
| | TACTCTGCGTTGATACCACTGCTT |
| bc90 | NBAAAAAAAAAAAAAAAAAAAAAAA |
| | AAAAAAANNNNNNNNAGTGTGCCAG |
| | TACTCTGCGTTGATACCACTGCTT |
| bc91 | NBAAAAAAAAAAAAAAAAAAAAAAA |
| | AAAAAAANNNNNNNNAACTTACCGG |
| | TACTCTGCGTTGATACCACTGCTT |
| bc92 | NBAAAAAAAAAAAAAAAAAAAAAAA |
| | AAAAAAANNNNNNNNGAATCAACCG |
| | TACTCTGCGTTGATACCACTGCTT |
| bc93 | NBAAAAAAAAAAAAAAAAAAAAAAA |
| | AAAAAAANNNNNNNNCAGTAGATGG |
| | TACTCTGCGTTGATACCACTGCTT |
| bc94 | NBAAAAAAAAAAAAAAAAAAAAAAA |
| | AAAAAAANNNNNNNNCTCCACTAGG |
| | TACTCTGCGTTGATACCACTGCTT |
| bc95 | NBAAAAAAAAAAAAAAAAAAAAAAA |
| | AAAAAAANNNNNNNNGGAGAGTGAG |
| | TACTCTGCGTTGATACCACTGCTT |
| bc96 | NBAAAAAAAAAAAAAAAAAAAAAAA |
| | AAAAAAANNNNNNNNGTCTTGTGGG |
| | TACTCTGCGTTGATACCACTGCTT |

TABLE 4
Klenow qPCR primer oligonucleotides.
These primers are used for confirming
Klenow extension of extension
oligonucleotides containing unique
spectral identifiers and UVISs by well
(as described in methods). Successful
well reactions confirm representation
of each spectral code in the resultant
combined pool.

| Oligonucleotide | Sequence |
|---|---|
| Forward primer | AAGCAGTGGTATCAACGCAGAGT |
| | (SEQ ID NO: 195) |
| Reverse primer | /5Cy5/NBAAAAAAAAAAAAAAA |
| | AAAAAAAAAAAAAAA |
| | (SEQ ID NO: 196) |

All publications, patents, and patent applications cited herein are hereby incorporated by reference with respect to the material for which they are expressly cited.

What is claimed is:

1. A method of analyzing multiple parameters in a single cell, the method comprising:
(i) generating an arrayed configuration comprising a plurality of single-cell analysis compartments, wherein each single cell analysis compartment comprises:
(a) a single cell and
(b) a single microbead having a lanthanide spectral signature, and immobilized to said microbead, a plurality of capture oligonucleotides that each comprise a first and a second domain, where the first domain comprises a spectral signature identifier sequence specific for the lanthanide spectral signature of the single microbead, and the second domain comprises a capture nucleic acid sequence that is substantially complementary to a sequence of one or more target polynucleotides in the cell;
wherein the lanthanide spectral signature of each microbead present in each single-cell analysis compartment of the plurality of single-cell analysis compartments is distinguishable from the lanthanide spectral signature of the microbeads in the other single-cell analysis compartments, and the capture oligonucleotide and the spectral signature of the microbead in each single-cell analysis compartment are unambiguously paired such that the microbead can be identified based on the spectral signature identifier sequence;
(ii) imaging the arrayed configuration to determine spatial position of the single cell and the microbead contained in each single-cell analysis compartment;
(iii) for at least some of the compartments, determining the spectral signature of the microbead contained therein;
(iv) analyzing a first parameter of the cell contained therein, wherein analyzing comprises detecting a signal from the single-cell analysis compartment and/or recording an image of the single cells contained in at least a portion of the single cell analysis compartments;
(v) for at least some of the single-cell analysis compartments in (iii) maintaining the single-cell analysis compartments under conditions in which the capture oligonucleotides hybridize to said one or more target polynucleotides;
(vi) analyzing a second parameter of the single cell wherein the second parameter comprises determining the sequence at least a portion of one or more target polynucleotides; and
(vii) determining the sequence of at least a portion of a capture oligonucleotide, where the sequence is sufficient to unambiguously pair the capture oligonucleotide with the microbead in the single-cell analysis compartment.

2. The method of claim 1, wherein the contents of multiple single cell analysis compartments are combined before claim step (vi).

3. The method of claim 1, wherein step (iv) comprises performing an image-based assay.

4. The method of claim 3, wherein the image-based assay comprises detecting a signal from a reagent employed in the image-based assay.

5. The method of claim 1, wherein steps (ii) and (iii) are performed concurrently.

6. The method of claim 1, wherein step (ii) and step re performed after step v).

7. The method of claim 1, wherein the array is an ordered array.

8. The method of claim 1, wherein the signal is a chemiluminescent signal or a fluorescent signal.

9. The method of claim 1, wherein each single cell analysis compartment further comprises reagents to assay the first parameter.

10. The method of claim 9, wherein the first parameter is expression of a secreted protein or molecule expressed on the cell surface.

11. The method of claim 10, wherein the reagents to assay the secreted protein or marker expressed on the cell surface comprises one or more antibodies to detect the molecule expressed on the cell surface or the secreted protein.

12. The method of claim 9, wherein reagents comprise intracellular dyes or stains.

13. The method of claim 9, wherein the first parameter is lipid or carbohydrate content of the single cell.

14. The method of claim 1, wherein the single cell analysis compartments comprise a single cell and the first parameter is cell morphology or cell motility.

15. The method of claim 14, wherein step (ii) is performed two or more times over desired intervals of time.

16. The method of claim 1, wherein the second parameter is expression of the one or more target polynucleotides.

17. The method of claim 1, further comprising a step of lysing the single cell in at least a portion of the single cell-analysis compartments performed after step (ii).

18. The method of claim 1, further comprising a step of collecting the microbeads bound to one or more target polynucleotides from the single-cell compartments.

19. The method of claim 1, further comprising a step of processing the one or more target polynucleotides bound to the microbeads to generate a reaction product.

20. The method of claim 19, wherein the processing step comprises an amplification reaction.

21. The method of claim 1, wherein the one or more target polynucleotides is genomic DNA, chromatin, or RNA.

22. The method of claim 1, wherein the one or more target polynucleotides is mRNA.

23. The method of claim 22, wherein the capture domain comprises a poly(T) tract that hybridizes to mRNA.

24. The method of claim 22, wherein the capture oligonucleotide further comprises a unique molecular identifier nucleic acid sequence.

25. The method of claim 24, further comprising a step of collecting the microbeads from the single-cell analysis compartments following step (v).

26. The method of claim 25, further comprising a step of processing the mRNA for RNA-Sect analysis.

27. The method of claim 26, wherein the processing step comprises a reverse transcriptase reaction to produces cDNA and subsequent amplification of the cDNA.

28. The method of claim 21, wherein the one or more target polynucleotides comprise genomic DNA and the method further comprises a step of processing the genomic DNA for genotyping or methylation profiling.

29. A method of analyzing multiple parameters in a single cell, the method comprising:
(i) generating an arrayed configuration comprising a plurality of single-cell analysis compartments, wherein each single cell analysis compartment contained in the plurality of single-cell analysis compartments comprises:
(a) a single cell, and
(b) a single microbead having a lanthanide spectral signature, and immobilized on said microbead, a plurality of capture oligonucleotides that each comprises a first and a second domain, where the first domain comprises a nucleic acid sequence specific to the lanthanide spectral signature and the second domain comprises a nucleic acid sequence that is substantially complementary to a target mRNA population from the cell;
wherein the lanthanide spectral signature of a microbead present in one single-cell analysis compartment is distinguishable from the lanthanide spectral signature of other microbeads in the other single cell analysis compartments, and the capture oligonucleotide and microbead in each single-cell analysis compartment are unambiguously paired;
(ii) imaging the arrayed configuration to determine spatial position of the cell and the microbead contained in each single-cell analysis compartment;
(iii) for at least some compartments, determining the spectral signatures of each microbead in each single cell analysis compartment;
(iv) analyzing a first parameter of the cell contained there, wherein analyzing comprises detecting a signal from the single-cell analysis compartment; and/or recording a microscope image of the single cell contained in at least a portion of the single cell analysis compartments;
(v) maintaining at least some of the single cell analysis compartments in (iii) in which the capture oligonucleotide captures the target mRNA population; and
(vi) recovering the microbeads hybridized to the target mRNA population;
(vii) processing the target mRNA population for RNA-Seq; and
(vii) performing RNA-Sect analysis.

30. The method of claim 29, wherein the second domain comprises a poly(T) tract.

31. The method of claim 29, wherein each single cell analysis compartment further comprises an assay reagent for analyzing the first parameter.

32. The method of claim 31, wherein the assay reagent comprises one or more antibodies.

33. The method of claim 29, wherein analyzing the first parameter comprises: analyzing: expression of one or more proteins, cell motility, cellular lipid content, cellular carbohydrate content, cell morphology, cytoskeleton structure, or functional response to selective perturbation or differential drug treatment.

34. The method of claim 33, wherein the one or more proteins comprise secreted proteins, cell surface proteins, or intracellular proteins.

35. The method of claim 33, wherein the first parameter step (ii) is performed two or more times over a desired interval of time.

36. The method of claim 29, wherein step (ii) and (iii) are performed at the same time.

37. The method of claim 29, wherein the capture oligonucleotide further comprises a unique molecular identifier nucleic acid sequence.

38. The method of claim 29, further comprising a step of collecting the microbeads from the single-cell analysis compartments following step (v).

39. The method of any one of claims 1 to 33, wherein the arrayed configuration is generated in a microwell device comprising an array of microwells.

40. The method of claim 39, wherein the microwell device comprises a clamping slide and the array of microwells.

41. The method of claim 40, wherein the microwell device further comprises an electrode slide.

42. The method of claim 39, wherein the microwell device comprises a microfluidic device.

43. The method of claim 39, wherein the microwell-size is about 80 to about 1,000 microns.

44. The method of any one of claims 1 to 33, wherein the arrayed configuration is generated by droplets deposited in individual wells of a microwell device or deposited in a defined pattern on a slide.

45. The method of any one of claims 1 to 44, wherein the cell single cell or nucleic in the single cell analysis compartment is a mammalian cell or yeast cell.

46. The method of claim 45, wherein the mammalian cell is a cancer cell or a lymphocyte.

47. An arrayed configuration comprising a plurality of single-cell analysis compartments, wherein each single-cell analysis compartment contained in the plurality of single-cell analysis compartments comprises:
(a) a single cell and
(b) a single microbead having a lanthanide spectral signature, and immobilized on said microbead, a plurality of capture oligonucleotides that comprises a first and a second domains, where the first domain comprises a nucleic acid sequence specific to the microbead and the second domain comprises a nucleic acid sequence that is substantially complementary to one or more target polynucleotides from the cell;
wherein the lanthanide spectral signature of a microbead present in one single-cell analysis compartment is distinguishable from the lanthanide spectral signature of other microbeads in the other single cell analysis compartments, and the capture oligonucleotide and lanthanide spectral signature of each microbead in each single-cell analysis compartment are unambiguously paired.

48. The arrayed configuration of claim 47, wherein each single-cell analysis compartment of the plurality of single-cell analysis compartments comprises assay reagents.

49. The arrayed configuration of claim 47 or 48, wherein the capture oligonucleotide further comprises a unique molecular identifier sequence.

50. The arrayed configuration of any one of claims 47 to 49, wherein the arrayed configuration comprises a microwell device comprising an array of microwells.

51. The arrayed configuration of claim 50, wherein the microwell device comprises a clamping slide and the array of microwells.

52. The arrayed configuration of claim 51, wherein the microwell device further comprises an electrode slide.

53. The arrayed configuration of any one of claims 47 to 52, wherein the arrayed configuration comprises at least 1000 different lanthanide spectral signatures.
